(19) United States
(12) Patent Application Publication
(10) Pub. No.: US 2023/0321226 A1
(43) Pub. Date: Oct. 12, 2023

(54) SYNTHETIC ARCHAEAL DIETHER LIPIDS

(71) Applicants: SORBONNE UNIVERSITE, Paris (FR); CENTRE NATIONAL DE LA RECHERCHE SCIENTIFIQUE, Paris (FR); INSERM (INSTITUT NATIONAL DE LA SANTÉ ET DE LA RECHERCHE MÉDICALE), Paris Cedex 13 (FR); APHP (ASSISTANCE PUBLIQUE - HÔPITAUX DE PARIS), Paris (FR); UNIVERSITÉ D’ORLÉANS, Orléans (FR); ÈCOLE NATIONALE SUPERIEUDRE DE CHIMIE DE RENNES (ENSCR), Rennes Cedex 7 (FR)

(72) Inventors: François LEMOINE, Montrouge (FR); Véronique MATEO, Vitry-sur-Seine (FR); Patrick MIDOUX, Saint Denis de l’Hôtel (FR); Chantal PICHON, Saint Denis de l’Hôtel (FR); Thierry BENVEGNU, Rennes (FR); Loïc LEMIÈGRE, Rennes (FR)

(21) Appl. No.: 16/308,103

(22) PCT Filed: Jun. 9, 2017
(86) PCT No.: PCT/FR2017/051481

§ 371 Date: Dec. 7, 2018

(30) Foreign Application Priority Data
Jun. 9, 2016 (FR) 1655296

(51) Int. Cl. A61K 39/39; C07H 15/08; A61K 9/127; A61K 39/00; A61P 35/00

(52) U.S. Cl. CPC A61K 39/39; A61K 9/1272; A61K 39/0011; A61P 35/00; C07H 15/08; A61K 2039/55555

(57) ABSTRACT

Disclosed are new synthetic compounds that includes a lipid diether to which a sugar group is grafted via a PEG spacer, and to now liposomes including at least one of the compounds. In particular, new synthetic compounds of general formula (I) and to new liposomes including at least one of the compounds is disclosed, as well as new liposomes for use as vectors and/or adjuvants, especially for use in vaccines.

### INCORPORATION BY REFERENCE

The file named “557 U.S. Corrected sequence listing” created on Jul. 12, 2019 with size of 3.16 KB, is hereby incorporated by reference into this specifciation.

### FIELD OF THE INVENTION

The present invention relates to the field of liposomes, the lipid compounds thereof and the pharmaceutical uses thereof. In particular the present invention relates to new synthetic compounds and the formulation of new liposomes comprising at least one of these compounds for use in vaccination.

### BACKGROUND OF INVENTION

Developing new compounds that have advantageous properties for the preparation of liposomes represents a major stake in medical research. Liposomes form a very effective way for delivering a molecule of interest inside cells. Liposomes represent for example an alternative to viral vectors for the delivery of nucleic acids. They also are of major therapeutic interest in cancerology. In addition to being used as vectors for drugs, liposomes are indeed developed for a therapeutic vaccination aiming to induce an immune response against tumour cells.

So-called conventional liposomes are mainly comprised of phospholipids similar to those present in the membranes of bacteria or of eukaryotic cells. The medical use of these liposomes can however be limited by stability problems and targeting problems. In order to be able to be administered orally or through blood, liposomes must indeed be able to resist an acid environment and/or interactions with the proteins and lipoproteins of the blood. Moreover, liposomes must be recognised by the cells for which the molecules that they transport are intended. In particular liposomes used as vaccination vectors must be recognised and internalised by the antigen-presenting cells so that the latter can induce a specific immune response to the antigen delivered by the liposomes.

The problem of liposome stability can be resolved by the use of lipids called ether lipids or archaeolipids. These lipids, naturally present in the membranes of archaebacteria, are characterised by the presence of ether bonds linking the phytanyl aliphatic chains that they are comprised of to a glycerol. The lipids present in the membranes of archaebacteria are therefore generally diethers or tetraethers of phytanols. The structure of the ether lipids plays an important role in the resistance of archaebacteria to extreme conditions. Likewise, the liposomes comprising these lipids, also called archaeosomes, generally display an increased stability to oxidative stress, to high temperatures, to acidic or alkaline conditions, to the action of phospholipases, bile salts and serum proteins (Patel GB, Sprott GD. Archaeobacterial ether lipid liposomes (archeosomes) as novel vaccines and drug delivery systems. Crit Rev Biotechnol 1999; 19: 317-357). WO9308202 thus describes archaeosomes that display an increased stability, obtained using total polar lipids extracted from archaebacteria. CA2269502 describes lipids derived from tetraethers and liposomes that contain them. WO2006061396 describes synthetic tetraether lipids similar to those present in the membranes of archaebacteria and liposomes that integrate these tetraether lipids. In addition to their increased stability, archaeosomes also have the advantage of having an intrinsic adjuvant effect, independent of any molecule that they can transport. WO0126683 thus describes the in vitro and in vivo activation of mouse macrophages and of dendritic cells by empty archaeosomes comprised of total polar lipids extracted from archaebacteria.

The problem of addressing liposomes can be resolved by the use of compounds that contain a lipid bound to a polar group that can be recognised by receptors expressed on the surface of targeted cells. Thus, liposomes comprising such compounds having a sugar polar group can be recognised by the lectin receptors of antigen-presenting cells (Benvegnu et al., Glycolipid-based nanosystems for the delivery of drugs, genes and vaccine adjuvant applications, Carbohydr. Chem., 2014, 40: 341-377). In particular, Espuelas et al., describe the addition of one, two or four mannose group(s) via a PEG spacer to a synthetic dioleylglycerol (DOG) lipid having two non-phytanyl aliphaticchains linked to a glycerol via an ether bond (Espuelas et al., Influence of ligand valency on the targeting of immature human dendritic cells by mannosylated liposomes, Bioconjugate Chem. 2008, 19:2385-2393). Espuelas et al., show that liposomes containing these compounds can target and be bound by human immature dendritic cells but are not able on their own to induce the expression of surface markers CD83, CD86 and HLA-DR of these cells. The compounds described by Espuelas et al., therefore do not confer any intrinsic adjuvant effect to the liposomes that contain them. WO2007112567 describes hemisynthetic diethers or tetraethers lipids comprising only phytanyl chains obtained from extracts of total polar lipids of archaebacteria. After isolation, these lipids are attached to sugar groups and the compounds obtained are used in the preparation of archaeosomes.

Obtaining hemisynthetic compounds comprising a diether lipid extracted from archaebacteria requires setting up archaebacteria cultures, extraction and isolation methods that are difficult to implement on a large scale. It is therefore necessary to be able to obtain synthetic compounds comprising diether lipids bound to a sugar group that have the advantageous properties of archaebacteria lipids.

The present invention thus relates to synthetic compounds comprising a diether lipid linked to a sugar group and liposomes comprising these compounds. The compounds of the invention are characterised by the presence of a branched aliphatic chain and of an unsubstituted linear aliphatic chain and by the presence of a glycoside group grafted to the diether lipid via a PEG spacer. In an unprecedented manner, the synthetic compounds of the invention provide the liposomes that contain them an in vitro and in vivo intrinsic adjuvant effect.

### SUMMARY

The present invention thus relates to a compound of formula I:

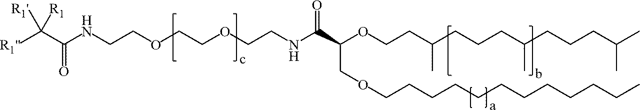

wherein:
a is an integer from 1 to 9;
b is an integer from 1 to 3;
c is an integer from 1 to 130, preferably from 5 to 20;
R₁, R₁′ and R₁″ are identical or different, each one independently representing an H, or a group of the type:

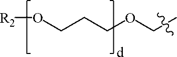

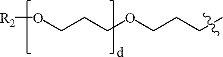

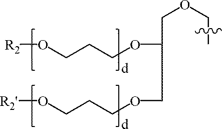

wherein R₂ and R₂′ are identical or different, each one independently representing an H or a sugar residue chosen from the list comprising mannose, glucose, fucose, oligomannoses comprising from 2 to 10 mannose units, glycans terminated with a mannose, glycans terminated with a fucose, Lewis-A trisaccharide 3′-sulfate, Lewis-B trisaccharide (Le^{b}), Lewis-X trisaccharide (Le^{x}), tri-N-acetylglucosamine (tri-GlcNAc), PIMs (phosphatidylinositol mannosides), in particular PIM₁ to PIM₆ (phosphatidylinositol mono- to hexa-mannoside), Man9GlcNAc2 oligosaccharide, N-linked oligosaccharides rich in mannose, α-fucose-1-4GlcNAc oligosaccharide, lacto-N-fucopentaose III oligosaccharide containing the Le^{x} trisaccharide, GlcNAc2 Man 3 oligosaccharide, Man 4 oligosaccharides, Manα1-3(Manα1-6)Manα1 oligosaccharide, lipoarabinomannans (LAMs), mannosylated lipoarabinomannans (ManLAMs);
d is an integer from 0 to 5;
with the condition that at least one of the R₂ and R₂′ groups is different from H and that R₂ is different from H when R₂′ is absent;
with the condition that at least one of the R₁, R₁′ and R₁″ groups is different from H.

According to an embodiment, the present invention relates to a compound of formula I:

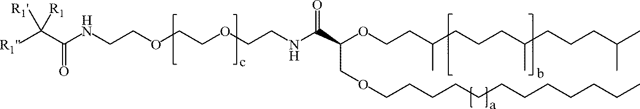

wherein:
a is an integer from 1 to 9;
b is an integer from 1 to 3;
c is an integer from 1 to 130, preferably from 5 to 20;
R₁, R₁′ and R₁″ are identical or different, each one independently representing an H or the group:

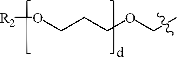

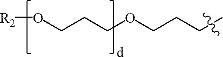

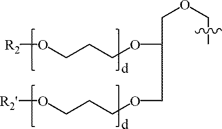

wherein d is an integer from 0 to 5;
wherein R₂ and R₂′ are identical or different, each one independently representing mannose, glucose, fucose, or oligomannoses comprising from 2 to 10 mannose units,
with the condition that at least one of the R₁, R₁′ and R₁″ groups is different from H.

According to an embodiment, the present invention relates to a compound of formula I:

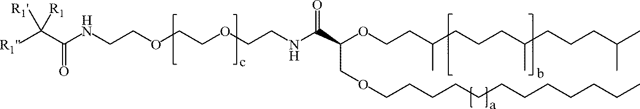

wherein:
a is an integer from 1 to 9;
b is an integer from 1 to 3;
c is an integer from 1 to 130, preferably from 5 to 20;
R₁, R₁′ and R₁″ are identical or different, each one independently representing an H or the group:

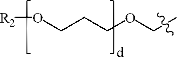

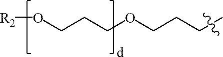

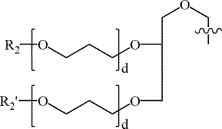

wherein d is an integer from 0 to 5;
wherein R₂ and R₂′ represent the group: 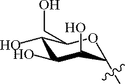
with the condition that at least one of the R₁, R₁′ and R₁″ groups is different from H.

The present invention also relates to a compound of formula II: 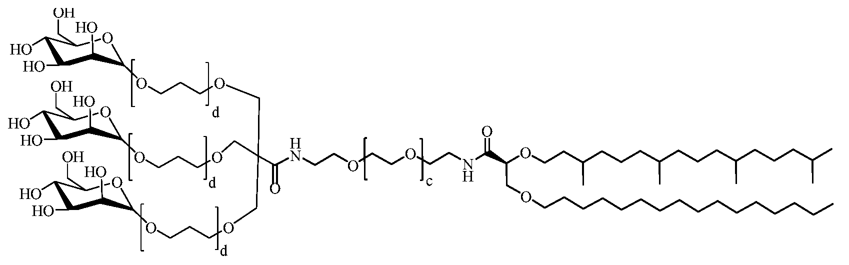 wherein:
c is an integer from 1 to 130, preferably from 5 to 20;
d is an integer from 1 to 5, preferably 2.

According to an embodiment, the present invention relates to the compound of formula II: 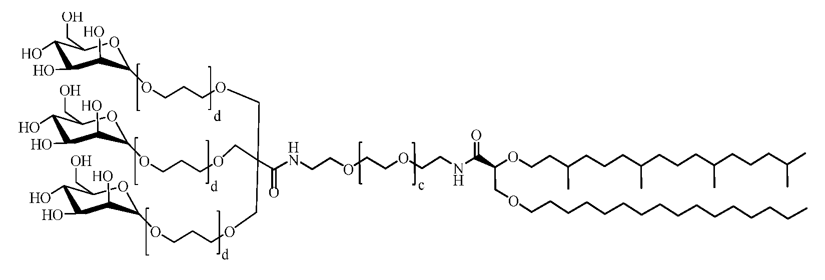 wherein:
c is 5;
d is 2.

The present invention also relates to a liposome comprising at least one compound according to the invention. According to an embodiment, the liposome of the invention comprises at least one compound of the invention in proportions between from about 1% to about 15% in mole percent with respect to the total number of moles of lipids, preferably from about 2% to about 10%, more preferably of about 5%.

The present invention also relates to a liposome of the invention further comprising at least one molecule of interest.

According to an embodiment, the molecule of interest contained in the liposome of the invention is able to induce an immune response.

According to an embodiment, the molecule of interest contained in the liposome of the invention is a nucleic acid.

According to an embodiment, the molecule of interest contained in the liposome of the invention is a cancer-associated antigen or a nucleic acid encoding a cancer-associated antigen.

According to an embodiment, the molecule of interest is a cancer-associated antigen selected from the group comprising: CAP-1, CD 4/m, cell surface proteins of the claudin family CLAUDIN-6, CLAUDIN-18.2 and CLAUDIN-12, c-myc, CT, GnT-V, HAGE, HAST-2, LAGE, NF1, NY-BR-1, proteinase 3, SAGE, SCGB3A2, SCP1, SCP2, SCP3, SSX, SURVrVin, TPI/m, TPTE, CDK4 (cyclin-dependent kinase 4), plS1″1′4′3, p53, AFP, β-catenin, caspase 8, mutated version of p21Ras, Bcr-abl chimera, MUM-I MUM-2, MUM-3, ELF2M, HSP70-2M, HST-2, KIAA0205, RAGE, myosin/m, 707-AP, CDC27/m, ETV6/AML, TEL/Amll, Dekcain, LDLR/FUT, Pml-RARαTEL/AMLI, NY-ESO-I, members of the MAGE family (Melanoma-associated antigen) MAGE-Al, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-B, MAGE-C, BAGE, DAM-6, DAM-10, members of the GAGE family (G antigen) GAGE-1, GAGE-2, GAGE-3, GAGE-4, GAGE-5, GAGE-6, GAGE-7B, GAGE-8, NA-88A, CAG-3, RCC-associated antigen G250, oncoproteins E6 and E7 derived from HPV (human papilloma virus), Epstein Barr virus antigens EBNA2-6, LMP-I, LMP-2, gp77, gp100, MART-1/Melan-A, tyrosinase, TRP-I and TRP-2 (tyrosinase-related protein), TRP-2-INT2, PSA, PSM, MC1R, ART4, CAMEL, CEA, CypB, HER2/neu, hTERT, hTRT, iCE, Mucl, Muc2, PRAME RU1, RU2, SART-I, SART-2, SART-3, WT and WT1, or a nucleic acid encoding said cancer-associated antigen.

The present invention also relates to a compound according to the invention for use as a vaccine adjuvant. Furthermore, the present invention relates to a liposome comprising at least one compound according to the invention for use as a vaccine adjuvant.

The present invention also relates to a liposome comprising at least one compound of the invention and further comprising at least one molecule of interest capable of inducing an immune response, for use as a vaccine.

The present invention also relates to a pharmaceutical composition comprising the compound according to the invention and at least one pharmaceutically acceptable excipient. Furthermore, the present invention relates to the liposome according to the invention and at least one pharmaceutically acceptable excipient.

### DEFINITIONS

In the present invention, the following terms are defined in the following way:
“Adjuvant” refers to a molecule that stimulates the immune response to an antigen and/or that modulates it in such a way as to obtain the expected response. In particular, the addition of adjuvants in vaccine formulations has for purpose to improve, accelerate and prolong the specific immune response directed against the antigen(s) comprised in these vaccine formulations. The advantages of adjuvants include the improvement in the immunogenicity of the antigens, the modification in the nature of the immune response, the reduction in the quantity of antigen(s) required to induce an effective immunisation, the reduction in the frequency of booster immunisations, and the improvement in the immune response in elderly subjects and immunocompromised subjects.
“Antigen” refers to any molecule that can initiate in a subject a cellular and/or humoral immune response.
“Dendritic cells” refers to antigen-presenting cells of the immune system that have in certain conditions cytoplasmic processes called dendrites. Dendritic cells have in particular for function to trigger the adaptive immune response induced in response to an antigen.
“Diether lipid” relates in the present invention to a glycerol whereon two alcohol functions are engaged in ether bonds with lipids.
“About” placed in front of a number, means 10% more or less of the nominal value of this number.
“Pharmaceutically acceptable excipient” relates to a vehicle or an inert support used as a solvent or diluent wherein the pharmaceutically active agent is formulated and/or administered, and which does not produce an undesirable, allergic or other reaction when it is administered to an animal, preferably a human. This includes all solvents, dispersion mediums, coatings, antibacterial and antifungal agents, isotonic agents, absorption-retarding agents and the like. For human administration, the preparations must satisfy standards concerning sterility, general safety and purity, such as required by the regulatory offices, such as for example the FDA (Food and Drug Administration) or the EMA (European Medicines Agency).
“Glycan” refers to a polymer comprised of monosaccharides connected together via a glycosidic bond.
“Glycoside” refers to a sugar group attached to another non-glucidic function via a glycosidic bond. The sugar group, also called sugar residue, can be a simple sugar (a monosaccharide) or comprise several sugars (an oligosaccharide or a polysaccharide).
“Immunogenic” relates to a molecule that induces an immune response in the subject to whom it is administered.
“Lectin” relates to a protein that is specifically and irreversibly bonded to certain glucides. Among the lectins, those present on the surface of certain immune cells are type C lectins.
“Lipid” relates to a saturated, unsaturated or polyunsaturated, linear or branched carbon chain.
“Lipoplex” relates to a liposome nucleic acid complex, in particular a cationic liposome nucleic acid complex.
“Lipopolyplex” relates to a liposome polycation nucleic acid ternary complex. When the nucleic acid is a DNA, the lipopolyplex is called LPD (Lipid Polycation - DNA). When the nucleic acid is an RNA, the lipopolyplex is called LPR (Lipid -Polycation - RNA).
“Liposome” relates to a vesicle formed of a bilayer of amphiphilic lipids enclosing an aqueous medium. The polar heads of the amphiphilic lipids are grouped together and are directed either toward the external aqueous medium, or toward the internal aqueous medium. The hydrophobic tails are buried inside the bilayer in such a way as to minimise their interaction with an aqueous medium.
“Adaptive immune response” relates to, after the administration of a vaccine, the development in a subject of a specific cellular and/or humoral immune response (mediated by antibodies). The adaptive immune response generally includes, without being limited to, one or several of the following effects: production of antibodies, of B lymphocytes, of T-helper cells and/or of cytotoxic T lymphocytes, specifically directed against one or several antigen(s) included in the vaccine. Preferably, the vaccinated subject will develop a protective or therapeutic adaptive immune response such that its/his/her resistance to an infection will be increased and/or the severity of the disease will be reduced.
“Sugar” in the present invention, may refer to a simple sugar or to a polymer of simple sugars. Simple sugars, also called oses or monosaccharides, are polyhydroxylated aldehydes or polyhydroxylated ketones. Glucose, mannose, ribulose and fructose area few examples of simple sugars. Among polymers of simple sugars, a distinction may be made between oligosaccharides, comprising from 2 to 20 ose residues, and polysaccharides, comprising more than 20 ose residues.
“Subject” refers to an animal, preferably a human being. In the meaning of the present invention, a subject may be a patient, namely a person receiving medical care, undergoing or having undergone medical treatment, or monitored within the framework of the development of a disease.
“Vaccine” relates to any preparation comprising a substance or a group of substances inducing in a subject an immune response directed against an infectious agent, for example a bacterium (for example type b Hæmophilus influenzæ (Hib), Streptococcus pneumoniæ, Neisseria meningitidis, Corynebacterium diphtheriæ, Clostridium tetani, Bordatella pertussis, Vibrio choleræ, Salmonella typhi) or a virus (for example the influenza virus (flu), chickenpox, hepatitis A, hepatitis B, human immunodeficiency virus (HIV), papillomavirus or poliovirus), or against a cancerous tumour. Prophylactic vaccines are administered in order to prevent a subject from contracting a disease or for attenuating the attack if the disease is contracted. Such vaccines generally comprise either an infectious agent (inactivated, or alive but attenuated), or fragments of an infectious agent (such as surface molecules coming from the infectious agent) or toxins produced by the infectious agent, obtained via purification or by genetic engineering. Therapeutic vaccines are intended for the treatment of specific diseases, such as, for example, cancers. Such vaccines comprise one or several tumour antigens and aim in particular to induce a cellular immune response, mediated by the T lymphocytes, directed against the tumour that is expressing this or these antigens.

### DETAILED DESCRIPTION

The present invention relates to a compound comprising:
two linear and/or branched aliphatic chains and of the same length, each one connected to a glycerol via an ether bond, characterised in that one of the aliphatic chains is a branched chain wherein the number of carbons of the linear chain is between 12 and 20, preferably between 16 and 20, with this number not including the branched carbons, and the other aliphatic chain is an unsubstituted linear chain wherein the number of carbons is between 12 and 20, preferably between 16 and 20;
a hydrophilic group chosen from the list comprising glycans terminated with a mannose, glycans terminated with a fucose, Lewis-A trisaccharide 3′-sulfate, Lewis-B trisaccharide (Le^{b}), Lewis-X trisaccharide (Le^{x}), tri-N-acetylglucosamine (tri-GlcNAc), PIMs (phosphatidylinositol mannosides), in particular PIM₁ to PIM₆ (phosphatidylinositol mono- to hexa-mannoside), Man9GlcNAc2 oligosaccharide, N-linked oligosaccharides rich in mannose, α-fucose-1-4GlcNAc oligosaccharide, lacto-N-fucopentaose III oligosaccharide containing the Le^{x} trisaccharide, GlcNAc2 Man 3 oligosaccharide, Man 4 oligosaccharides, Manα1-3(Manα1-6)Manα1 oligosaccharide, lipoarabinomannans (LAMs), mannosylated lipoarabinomannans (ManLAMs) and antennery sugars comprising from 2 to 5 sugar residues characterised in that the antennery sugar residues are attached by an oligo(propylene glycol)n spacer wherein n is ranging from 1 to 5 and preferably n = 2, the sugar residues being chosen independently from the list comprising mannose, glucose, fucose, and oligomannoses comprising from 2 to 10 mannose units;
a [PEG]m spacer separating the hydrophilic group from the aliphatic chains wherein m is between 1 and 130, preferably between 5 and 20.

The present invention relates to a compound of formula I:

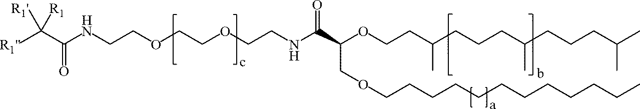

wherein:
a is an integer from 1 to 9;
b is an integer from 1 to 3;
c is an integer from 1 to 130, preferably from 5 to 20;
R₁, R₁′ and R₁″ are identical or different, each one independently representing an H, or a group of the type:

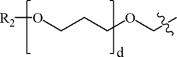

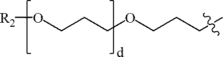

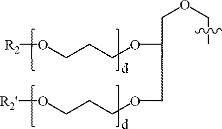

wherein R₂ and R₂′ are identical or different, each one independently representing an H or a sugar residue chosen from the list comprising mannose, glucose, fucose, oligomannoses comprising from 2 to 10 mannose units, glycans terminated with a mannose, glycans terminated with a fucose, Lewis-A trisaccharide 3′-sulfate, Lewis-B trisaccharide (Le^{b}), Lewis-X trisaccharide (Le^{x}), tri-N-acetylglucosamine (tri-GlcNAc), PIMs (phosphatidylinositol mannosides), in particular PIM₁ to PIM₆ (phosphatidylinositol mono- to hexa-mannoside), Man9GlcNAc2 oligosaccharide, N-linked oligosaccharides rich in mannose, α-fucose-1-4GlcNAc oligosaccharide, lacto-N-fucopentaose III oligosaccharide containing the Le^{x} trisaccharide, GlcNAc2 Man 3 oligosaccharide, Man 4 oligosaccharides, Manα1-3(Manα1-6)Manα1 oligosaccharide, lipoarabinomannans (LAMs), mannosylated lipoarabinomannans (ManLAMs);
d is an integer from 0 to 5;
with the condition that at least one of the R₂ and R₂′ groups is different from H and that R₂ is different from H when R₂′ is absent;
with the condition that at least one of the R₁, R₁′ and R₁″ groups is different from H.

According to an embodiment, the present invention relates to a compound of formula I:

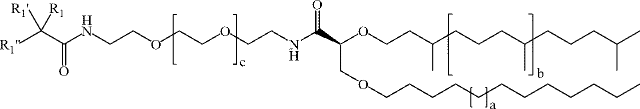

wherein:
a is an integer from 1 to 9;
b is an integer from 1 to 3;
c is an integer from 1 to 130, preferably from 5 to 20;
R₁, R₁′ and R₁″ are identical or different, each one independently representing an H or the group:

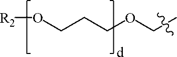

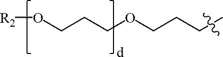

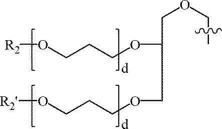

wherein R₂ and R₂′ are identical or different, each one independently representing an H or a sugar residue chosen from the list comprising mannose, glucose, fucose, oligomannoses comprising from 2 to 10 mannose units, glycans terminated with a mannose, glycans terminated with a fucose, Lewis-A trisaccharide 3′-sulfate, Lewis-B trisaccharide (Le^{b}), Lewis-X trisaccharide (Le^{x}), tri-N-acetylglucosamine (tri-GlcNAc), PIMs (phosphatidylinositol mannosides), in particular PIM₁ to PIM₆ (phosphatidylinositol mono- to hexa-mannoside), α-fucose-1-4GlcNAc oligosaccharide, lacto-N-fucopentaose III oligosaccharide containing the Le^{x} trisaccharide, GlcNAc2 Man 3 oligosaccharide, lipoarabinomannans (LAMs), mannosylated lipoarabinomannans (ManLAMs);
d is an integer from 0 to 5;
with the condition that at least of the R₂ and R₂′ groups is different from H and that R₂ is different from H when R₂′ is absent;
with the condition that at least two of the R₁, R₁′ and R₁″ groups are different from H.

According to an embodiment, the present invention relates to a compound of formula I:

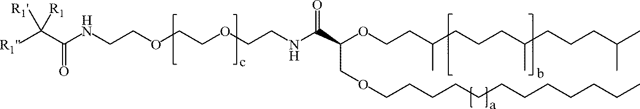

wherein:
a is equal to 5;
b is equal to 2;
c is equal to 5;
R₁, R₁′ and R₁″ are identical or different, each one independently representing an H or the group:

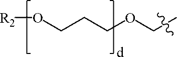

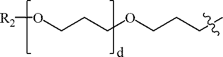

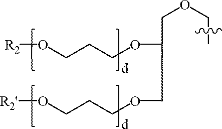

wherein R₂ and R₂′ are identical or different, each one independently representing an H or a sugar residue chosen from the list comprising mannose, glucose, fucose, oligomannoses comprising from 2 to 10 mannose units, glycans terminated with a mannose, glycans terminated with a fucose, Lewis-A trisaccharide 3′-sulfate, Lewis-B trisaccharide (Le^{b}), Lewis-X trisaccharide (Le^{x}), tri-N-acetylglucosamine (tri-GlcNAc), PIMs (phosphatidylinositol mannosides), in particular PIM₁ to PIM₆ (phosphatidylinositol mono- to hexa-mannoside), Man9GlcNAc2 oligosaccharide, N-linked oligosaccharides rich in mannose, α-fucose-1-4GlcNAc oligosaccharide, lacto-N-fucopentaose III oligosaccharide containing the Le^{x} trisaccharide, GlcNAc2 Man 3 oligosaccharide, Man 4 oligosaccharides, Manα1-3(Manα1-6)Manα1 oligosaccharide, lipoarabinomannans (LAMs), mannosylated lipoarabinomannans (ManLAMs);
d is an integer from 0 to 5;
with the condition that at least one of the R₂ and R₂′ groups is different from H and that R₂ is different from H when R₂′ is absent;
with the condition that at least one of the R₁, R₁′ and R₁″ groups is different from H.

According to an embodiment, the present invention relates to a compound of formula I:

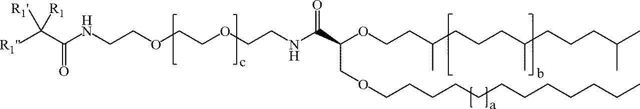

wherein:
a is an integer from 1 to 9;
b is an integer from 1 to 3;
c is an integer from 1 to 130, preferably from 5 to 20;
R₁, R₁′ and R₁″ are identical or different, each one independently representing an H or the group:

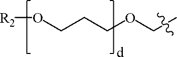

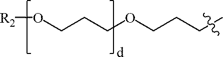

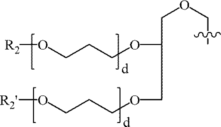

wherein d is an integer from 0 to 5;
wherein R₂ and R₂′ represent the group: 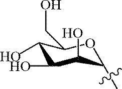
with the condition that at least one of the R₁, R₁′ and R₁″ groups is different from H.

According to an embodiment, the present invention relates to a compound of formula I:

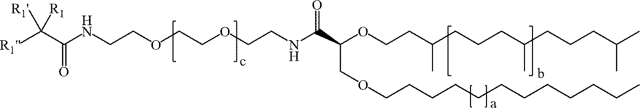

wherein:
a is equal to 5;
b is equal to 2;
c is equal to 5;
R₁, R₁′ and R₁″ are identical or different, each one independently representing an H or the group:

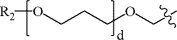

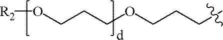

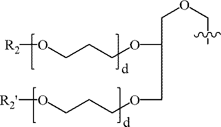

wherein d is equal to 2;
wherein R₂ and R₂′ represent the group: 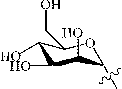
with the condition that at least one of the R₁, R₁′ and R₁″ groups is different from H.

According to an embodiment, the present invention relates to a compound of formula I:

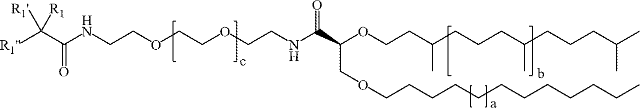

wherein:
a is equal to 5;
b is equal to 2;
c is equal to 5;
R₁, R₁′ and R₁″ represent the group:

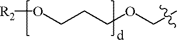

wherein d is equal to 2;
wherein R₂ represents the group:

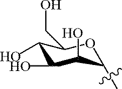 [SMILES: CCCCCCCC/C=C\CCCCCCCCOP(=O)(NCCn1cc[n+](C)c1)OCCCCCCCC/C=C\CCCCCCCC ; [I-]]

According to an embodiment, the present invention therefore relates to a tri-mannosylated compound of formula II: 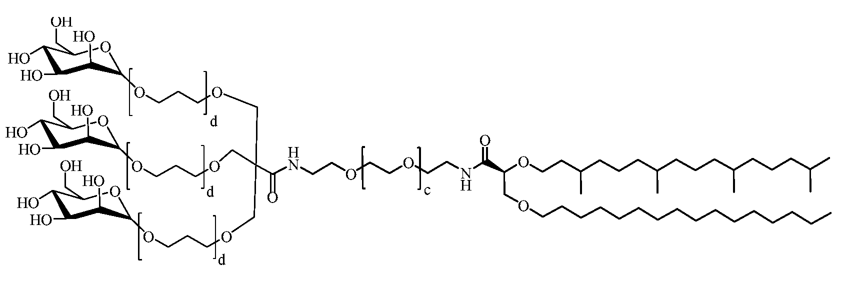 wherein:
c is an integer from 1 to 130, preferably from 5 to 20;
d is an integer from 1 to 5, preferably 2.

According to a particular embodiment, this invention relates to a tri-mannosylated compound of formula II: 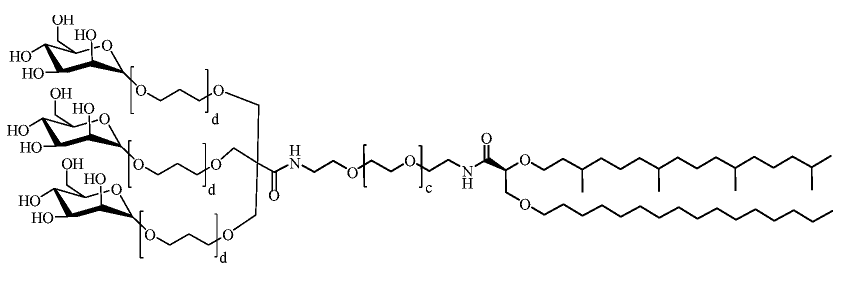 wherein:
c is an integer from 2 to 30, preferably from 3 to 20 and more preferably from 4 to 10;
d is an integer from 1 to 5, preferably 2.

According to a particular embodiment, the present invention relates to a tri-mannosylated compound of formula II: 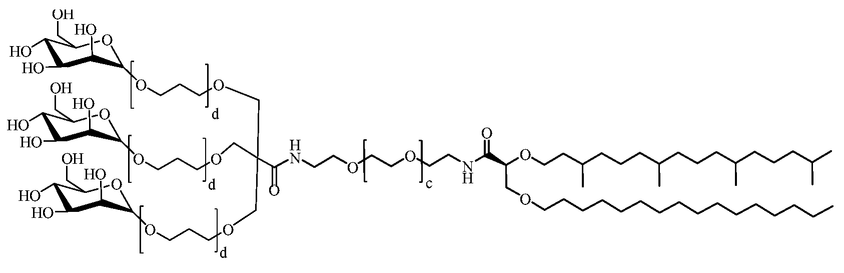 wherein:
c is 5;
d is 2.

The present invention relates to a synthetic compound comprising a lipid that has structural characteristics specific to the lipids naturally present in the membranes of archaebacteria.

Lipids naturally present in the membranes of archaebacteria are characterised by the presence of ether bonds connecting their aliphatic chains to a glycerol molecule. They are also characterised by the presence of phytanyl aliphatic chains that are regularly branched. The lipid ethers present in the membranes of archaebacteria are thus generally diethers of phytanols (such as archaeol) or tetraethers of phytanols (such as caldarchaeol). The glycerolipids present in the membranes of bacteria and of eukaryotic cells, referred to as conventional lipids, have ester bonds connecting their fatty acids to a glycerol molecule. Moreover, the fatty acids of these conventional lipids are generally saturated or unsaturated aliphatic chains, that do not comprise any branches. The structure of the lipid ethers present in the membranes of archaebacteria plays an important role in the resistance of archaebacteria to extreme conditions such as high temperatures or an acid environment.

The present invention relates to a synthetic compound comprising two ether bonds, connecting a branched aliphatic chain and an unsubstituted linear aliphatic chain to a glycerol. The present invention relates to a compound comprising a diether lipid.

According to an embodiment, the diether lipid of the invention comprises two aliphatic chains, of which the linear structures, not including the branched carbons, are of the same length, connected to a glycerol via an ether bond, one of the aliphatic chains being a branched chain and the other aliphatic chain being an unsubstituted linear chain.

In an embodiment, the aliphatic chains of the diether lipid of the invention are saturated. In another embodiment, the aliphatic chains of the diether lipid of the invention are unsaturated. In another embodiment, the branched aliphatic chain is saturated and the unsubstituted linear aliphatic chain is unsaturated. In another embodiment, the branched aliphatic chain is unsaturated and the unsubstituted linear aliphatic chain is saturated.

In an embodiment, the linear structure of the same length of the branched chain and the unsubstituted linear chain of the diether lipid of the invention comprises between 12 and 20 carbons, preferably between 16 and 20 carbons, and more preferably 16 carbons.

In an embodiment the linear structure of the same length of the branched chain and the unsubstituted linear chain of the diether lipid of the invention comprises 12, 13, 14, 15, 16, 17, 18, 19 or 20 carbons.

The present invention relates to a compound comprising a diether lipid to which a hydrophilic group, or polar group is grafted.

In an embodiment, the hydrophilic group grafted to the diether lipid of the invention is a glycoside.

In an embodiment, the hydrophilic group grafted to the diether lipid of the invention is a sugar recognised by lectins, proteins able of binding sugar residues, and preferably by type C lectins.

In an embodiment, the hydrophilic group grafted to the diether lipid of the invention is a sugar recognised by lectins DC-SIGN (CD209), DEC205/CD205, langerin (CD207), CLEC9A, CLEC4D, CD40, and/or the mannose receptor (MR).

According to an embodiment, the hydrophilic group grafted to the diether lipid of the invention is a sugar chosen from the list comprising glycans terminated with a mannose, glycans terminated with a fucose, Lewis-A trisaccharide 3′-sulfate, Lewis-B trisaccharide (Le^{b}), Lewis-X trisaccharide (Le^{x}), tri-N-acetylglucosamine (tri-GlcNAc), PIMs (phosphatidylinositol mannosides), in particular PIM₁ to PIM₆ (phosphatidylinositol mono- to hexa-mannoside), Man9GlcNAc2 oligosaccharide, N-linked oligosaccharides rich in mannose, α-fucose-1-4GlcNAc oligosaccharide, lacto-N-fucopentaose III oligosaccharide containing the Le^{x} trisaccharide, GlcNAc2 Man 3 oligosaccharide, Man 4 oligosaccharides, Manα1-3(Manα1-6)Manα1 oligosaccharide, lipoarabinomannans (LAMs), mannosylated lipoarabinomannans (ManLAMs) and the antennery sugars comprising from 2 to 5 sugar residues characterised in that the antennery sugar residues are attached by an oligo(propylene glycol)n spacer wherein n is ranging from 1 to 5 and preferably n = 2, the sugar residues being chosen independently from the list comprising mannose, glucose, fucose, and oligomannoses comprising from 2 to 10 mannose units.

According to an embodiment, the hydrophilic group grafted to the diether lipid of the invention is a sugar chosen from the list comprising glycans terminated with a mannose, glycans terminated with a fucose, Lewis-A trisaccharide 3′-sulfate, Lewis-B trisaccharide (Le^{b}), Lewis-X trisaccharide (Le^{x}), tri-N-acetylglucosamine (tri-GlcNAc), PIMs (phosphatidylinositol mannosides), in particular PIM₁ to PIM₆ (phosphatidylinositol mono- to hexa-mannoside), α-fucose-1-4GlcNAc oligosaccharide, lacto-N-fucopentaose III oligosaccharide containing the Le^{x} trisaccharide, GlcNAc2 Man 3 oligosaccharide, lipoarabinomannans (LAMs), mannosylated lipoarabinomannans (ManLAMs) and the antennery sugars comprising from 2 to 5 sugar residues characterised in that the antennery sugar residues are attached by an oligo(propylene glycol)n spacer wherein n is ranging from 1 to 5 and preferably n = 2, the sugar residues being chosen independently from the list comprising mannose, glucose, fucose, and oligomannoses comprising from 2 to 10 mannose units.

According to an embodiment, the hydrophilic group grafted to the diether lipid of the invention is an antennery sugar comprising from 2 to 5 sugar residues characterised in that the antennery sugar residues are attached by an oligo(propylene glycol)n spacer wherein n is ranging from 1 to 5 and preferably n = 2, the sugar residues being chosen independently from the list comprising mannose, glucose, fucose, and oligomannoses comprising from 2 to 10 mannose units.

According to an embodiment, the hydrophilic group grafted to the diether lipid of the invention is an antennery sugar comprising 2, 3, 4 or 5 sugar residues attached by an oligo(propylene glycol)n spacer wherein n is ranging from 1 to 5 and preferably n = 2, the sugar residues being chosen independently from the list comprising mannose, glucose, fucose, and oligomannoses comprising from 2 to 10 mannose units.

According to an embodiment, the hydrophilic group grafted to the diether lipid of the invention is an antennery sugar comprising 3 sugar residues attached by an oligo(propylene glycol)n spacer wherein n is ranging from 1 to 5 and preferably n = 2, the sugar residues being chosen independently from the list comprising mannose, glucose, fucose, and oligomannoses comprising from 2 to 10 mannose units.

According to an embodiment, the hydrophilic group grafted to the diether lipid of the invention is an antennery sugar comprising 2, 3, 4 or 5 mannoses attached by an oligo(propylene glycol)n spacer wherein n is ranging from 1 to 5 and preferably n = 2.

According to an embodiment, the hydrophilic group grafted to the diether lipid of the invention is an antennery sugar comprising 2, 3, 4 or 5 glucoses attached by an oligo(propylene glycol)n spacer wherein n is ranging from 1 to 5 and preferably n = 2.

According to an embodiment, the hydrophilic group grafted to the diether lipid of the invention is an antennery sugar comprising 2, 3, 4 or 5 fucoses attached by an oligo(propylene glycol)n spacer wherein n is ranging from 1 to 5 and preferably n = 2.

According to an embodiment, the hydrophilic group grafted to the diether lipid of the invention is an antennery sugar comprising 2, 3, 4 or 5 oligomannoses comprising from 2 to 10 mannose units attached by an oligo(propylene glycol)n spacer wherein n is ranging from 1 to 5 and preferably n = 2.

The present invention relates to a compound comprising a diether lipid which is grafted to the hydrophilic group, or polar group, by the intermediary of a polyethylene glycol spacer (PEG).

In an embodiment, the PEG spacer has the following structure:

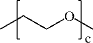

wherein c is between 1 and 130, preferably between 5 and 20.

According to an embodiment, c is equal to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

According to an embodiment, c is an integer ranging from 2 to 30, preferably from 3 to 20, and more preferably from 4 to 10. According to a particular embodiment, c is equal to 5.

The synthesis of the compound of the invention can be carried out by different routes known to those skilled in the art.

In an embodiment (described in Laine et al., Folate-equipped pegylated archaeal lipid derivatives: synthesis and transfection properties, Chem. Eur. J. 2008, 14, 83330-8340), a diether lipid according to the invention can be obtained from a glycerol derivative, itself synthesised from a phytol and from a (R)-solketal. The reaction of this glycerol derivative with hexadecyl triflate in the presence of a proton sponge makes it possible to obtain the corresponding diether. The hydrogenolysis of the benzyloxy group leads to alcohol which can then be converted into carboxylic acid or into amine. The diethers lipid thus obtained, having a carboxylic acid function or an amine function, may be used for the synthesis of compounds according to the invention.

In an embodiment, the synthesis of the tri-mannosylated compound of formula II is carried out according to a four-step scheme starting from carboxylic acid tri-mannosylated ligand and 1-O-carboxyl-2-O-Phytanyl-3-O-hexadecane-sn-glycerol referred to hereinafter as diether lipid having a carboxylic acid function (or carboxylic acid diether):
1) the carboxylic acid tri-mannosylated ligand is prepared by benzylation of pentaerythritol triallyl ether, followed by a hydroboration-oxidation/allylation/hydroboration-oxidation sequence, a trimannosylation of the free hydroxyls by a tetra-O-benzoylated trichloroacetimidate mannosyl donor, a deprotection of the hydroxyl of the pentaerythritol followed by an oxidation into carboxylic acid;
2) the peptide coupling between the carboxylic acid diether and the poly(ethylene glycol) amino-azide chain is carried out using an ester activated by a benzotriazole core (TBTU) in the presence of N,N′-diisopropylethylamine (DIEA), under an inert atmosphere in anhydrous dichloromethane;
3) after reduction of the azide function in Staudinger conditions, a second peptide coupling in the presence of the reagents TBTU and DIEA allows for a grafting of the carboxylic acid tri-mannosylated ligand;
4) a deprotection of the hydroxyls of mannose moieties in Zemplen conditions (MeONa, CH₂Cl₂/MeOH) leads to the final tri-mannosylated compound.

The present invention also relates to a liposome comprising at least one compound of the invention.

According to an embodiment, the liposome of the invention comprises at least one compound of the invention in proportions rangingfrom about 1% to about 15% in mole percent with respect to the total number of moles of lipids, preferably from about 2% to about 10%, more preferably of about 5%.

In an embodiment, the liposome of the invention comprises at least one compound of the invention in proportions ranging from about 1% to about 2%, 3%, 4%, 5%, 6%, 7%, 8%, 9%, 10%, 11%, 12%, 13%, 14%, or 15% in mole percent with respect to the total number of moles of lipids.

In an embodiment, the liposome of the invention comprises at least one compound of the invention in proportions ranging from about 1%, 2%, 3%, 4%, 5%, 6%, 7%, 8%, 9%, 10%, 11%, 12%, 13%, or 14%, to about 15% in mole percent with respect to the total number of moles of lipids.

In an embodiment, the liposome of the invention comprises about 1%, 2%, 3%, 4%, 5%, 6%, 7%, 8%, 9%, 10%, 11%, 12%, 13%, 14% or 15% of a compound of the invention in mole percent with respect to the total number of moles of lipids.

According to an embodiment, the liposome of the invention comprises at least one compound of the invention in proportions ranging from about 1% to about 15% by weight with respect to the total weight of the liposome, preferably from about 2% to about 10%, more preferably of about 5%.

In an embodiment, the liposome of the invention comprises about 1%, 2%, 3%, 4%, 5%, 6%, 7%, 8%, 9%, 10%, 11%, 12%, 13%, 14% or 15% of a compound of the invention, by weight with respect to the total weight of the liposome.

In an embodiment, the liposome of the invention comprises at least one compound according to the invention alone or in a mixture with one or several synthetic, hemisynthetic or natural lipids.

According to an embodiment, the liposome of the invention comprises at least one compound according to the invention and the lipid KLN25 (O,O-dioleyl-N-[3N-(Nmethylimidazoliumbromide)propylene] phosphoramidate) of formula:

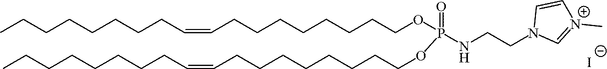

According to an embodiment, the liposome of the invention comprises at least one compound according to the invention and the lipid MM27 (O,O-dioleyl (-N-(histamine)phosphoramidate) of formula:

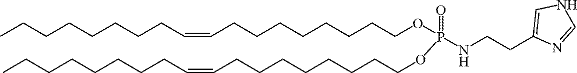

According to an embodiment, the liposome of the invention comprises at least one compound according to the invention, the lipid KLN25 and the lipid MM27.

According to an embodiment, the liposome of the invention comprises 5% of the compound according to the invention, 47.5% of lipid KLN25, and 47.5% of lipid MM27, in mole percent with respect to the total number of moles of lipids.

According to an embodiment, the liposome of the invention comprises at least one compound according to the invention and a cationic lipid. In an embodiment, the cationic lipid is a lipid that has one polar head consisting of an imidazolium group. In an embodiment, the liposome of the invention is a cationic liposome.

The preparation of the liposome of the invention can be carried out by different methods well-known to those skilled in the art. For example, the liposome of the invention can be prepared according to the method of hydrating a dry lipid film (Pichon C, Midoux P. (2013) Mannosylated and Histidylated LPR Technology for Vaccination with Tumor Antigen mRNA. Methods Mol Biol. 969:247-74).

According to an embodiment, the liposome of the invention comprises at least one molecule of interest. According to an embodiment, the liposome of the invention comprises at least one molecule of interest to be delivered in vitro or in vivo to a cell.

In an embodiment, the molecule of interest is comprised within the lipid bilayer of the liposome of the invention. In another embodiment, the molecule of interest is comprised within the hydrophilic compartment of the liposome of the invention.

In an embodiment, the molecule of interest is a nucleic acid, for example and without being limited to, the molecule of interest is a DNA, a RNA, a mRNA, a siRNA, or a microRNA. In another embodiment, the molecule of interest is a protein or a peptide. In another embodiment, the molecule of interest is a therapeutic agent. In another embodiment, the molecule of interest is a colouring agent or a marker.

In an embodiment, the molecule of interest is an immunogenic molecule. In an embodiment, the molecule of interest is able to induce an immune response.

In an embodiment, the molecule of interest is an antigen of bacterial origin, of fungal origin or of viral origin. In an embodiment, the molecule of interest is an antigen of bacterial origin chosen from the non-exhaustive list comprising the capsular polyoside (polyribosyl ribitol phosphate or PRP) of type b Hæmophilus influenzæ; polyoside serotypes 1, 2, 3, 4, 5, 6B, 7F, 8, 9N, 9V, 10A, 11A, 12F, 14, 15B, 17F, 18C, 19A, 19F, 20, 22F, 23F, 33F of Streptococcus pneumoniae; the polyoside of Neisseria meningitidis group A; the polyoside of Neisseria meningitidis group B; the capsular polyoside Vi of Salmonella typhi (Ty 2 strain).

In an embodiment, the molecule of interest is a cancer-associated antigen or a nucleic acid encoding a cancer-associated antigen.

In a preferred embodiment, the immunogenic molecule contained in the liposome of the invention is a tumour antigen or a nucleic acid encoding a tumour antigen. According to an embodiment, the tumour antigen can be chosen from the non-exhaustive list comprising: CDK4 (cyclin-dependent kinase 4), p1S^{1″1′4′3}, p53, AFP, β-catenin, caspase 8, mutated version of p21^{Ras}, Bcr-abl chimera, MUM-I MUM-2, MUM-3, ELF2M, HSP70-2M, HST-2, KIAA0205, RAGE, myosin/m, 707-AP, CDC27/m, ETV6/AML, TEL/Amll, Dekcain, LDLR/FUT, Pml-RARαTEL/AMLI, NY-ESO-I, members of the MAGE family (Melanoma-associated antigen) MAGE-Al, MAGE-A2, MAGE-A3, MAGE-A4, MAGE-A6, MAGE-10, MAGE-12, BAGE, DAM-6, DAM-10, members of the GAGE family (G antigen) GAGE-1, GAGE-2, GAGE-3, GAGE-4, GAGE-5, GAGE-6, GAGE-7B, GAGE-8, NA-88A, CAG-3, RCC-associated antigen G250, oncoproteins E6 and E7 derived from HPV (human papilloma virus), Epstein Barr virus antigens EBNA2-6, LMP-I, LMP-2, gp77, gp100, MART-1/Melan-A, tyrosinase, TRP-I and TRP-2 (tyrosinase-related protein), PSA, PSM, MC1R, ART4, CAMEL, CEA, CypB, HER2/neu, hTERT, hTRT, iCE, Muc1, Muc2, PRAME RU1, RU2, SART-I, SART-2, SART-3, and WT1. According to an embodiment, the tumour antigen can be chosen from the non-exhaustive list comprising: CAP-1, CD 4/m, cell surface proteins of the claudin family CLAUDIN-6, CLAUDIN-18.2 and CLAUDIN-12, c-myc, CT, GnT-V, HAGE, HAST-2, LAGE, NF1, NY-BR-1, proteinase 3, SAGE, SCGB3A2, SCP1, SCP2, SCP3, SSX, SURVrVin, TPI/m, TPTE, CDK4 cyclin-dependent kinase 4), p1S^{1″1′4′3}, p53, AFP, β-catenin, caspase 8, mutated version of p21^{Ras}, Bcr-abl chimera, MUM-I MUM-2, MUM-3, ELF2M, HSP70-2M, HST-2, KIAA0205, RAGE, myosin/m, 707-AP, CDC27/m, ETV6/AML, TEL/Amll, Dekcain, LDLR/FUT, Pml-RARαTEL/AMLI, NY-ESO-I, members of the MAGE family (Melanoma-associated antigen) MAGE-Al, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-B, MAGE-C, BAGE, DAM-6, DAM-10, members of the GAGE family (G antigen) GAGE-1, GAGE-2, GAGE-3, GAGE-4, GAGE-5, GAGE-6, GAGE-7B, GAGE-8, NA-88A, CAG-3, RCC-associated antigen G250, oncoproteins E6 and E7 derived from HPV (human papilloma virus), Epstein Barr virus antigens EBNA2-6, LMP-I, LMP-2, gp77, gp100, MART-1/Melan-A, tyrosinase, TRP-I and TRP-2 (tyrosinase-related protein), TRP-2-INT2, PSA, PSM, MC1R, ART4, CAMEL, CEA, CypB, HER2/neu, hTERT, hTRT, iCE, Mucl, Muc2, PRAME RU1, RU2, SART-I, SART-2, SART-3, WT and WT1.

According to an embodiment, the tumour antigen is chosen from the list comprising oncoproteins E6 and E7 derived from HPV (human papilloma virus), MART-1/Melan-A. According to another embodiment, the tumour antigen is the oncoprotein E7 derived from HPV (human papilloma virus).

In an embodiment, the molecule of interest is a polypeptide. In an embodiment, the polypeptide is of bacterial origin, of fungal origin or of viral origin.

In an embodiment, the molecule of interest is a polypeptide of bacterial origin corresponding to a protein, or to a fragment of this protein, chosen from the non-exhaustive list comprising XcpQ and PopB of Pseudomonas aeruginosa; factor H binding protein (fHBP), Neisseria Heparin Binding Antigen (NHBA) of Neisseria meningitidis group B; Neisseria adhesin A (NadA) of Neisseria meningitidis group B, diphtheria toxoid of Corynebacterium diphtheriæ; tetanus toxoid of Clostridium tetani; pertussis toxoid (whooping cough toxoid), filamentous hemagglutinin, pertactin of Bordatella pertussis; recombinant cholera toxin B subunit (rCTB) of Vibrio choleræ.

In an embodiment, the molecule of interest is a polypeptide of viral origin corresponding to a protein, or a fragment of this protein, chosen from the non-exhaustive list comprising the proteins GAG (p24, p17, p9 and p7), Pol (p64, p51, p10 and p32) and Env (gp41 and gp120) of the human immunodeficiency virus (HIV); pre-membrane precursor protein (preM) and M (for membrane) protein of the Zika virus; EDIII domain of the E protein of the dengue virus; fusion glycoproteins (or RSV-F for respiratory syncytial virus F protein) of human respiratory syncytial virus; haemagglutinin of the influenza type A virus H5N1 subtype; nuclear protein NP of the influenza virus; surface antigen of the hepatitis B virus; S and pre-S2 proteins of the hepatitis B virus; L1 protein of type 6 human papillomavirus (HPV); L1 protein of type 11 human papillomavirus (HPV); L1 protein of type 16 human papillomavirus (HPV); L1 protein of type 18 human papillomavirus (HPV); L1 protein of type 31 human papillomavirus (HPV); L1 protein of type 33 human papillomavirus (HPV); L1 protein of type 45 human papillomavirus (HPV); L1 protein of type 52 human papillomavirus (HPV); L1 protein of type 58 human papillomavirus (HPV).

In an embodiment, the liposome of the invention forms a lipoplex comprising at least one compound of the invention. In other terms, in an embodiment, the liposome of the invention comprises at least one nucleic acid.

The preparation of the lipoplex of the invention can be carried out by different methods well known to those skilled in the art. For example, the lipoplex of the invention can be obtained by adding a solution of nucleic acid to a preparation of liposomes according to the invention.

According to an embodiment, the lipoplex of the invention is obtained by mixing a solution comprising a nucleic acid, preferably an mRNA, encoding a tumour antigen to a preparation of liposomes according to the invention.

According to an embodiment, the lipoplex of the invention is obtained by mixing a solution comprising a nucleic acid, preferably an mRNA, encoding a tumour antigen to a preparation of liposomes comprising 5% of the compound according to the invention, 47.5% of lipid KLN25, and 47.5% of lipid MM27, in mole percent with respect to the total number of moles of lipids.

In an embodiment, the liposome of the invention forms a lipopolyplex comprising at least one compound of the invention. In other terms, in an embodiment, the liposome of the invention comprises at least one nucleic acid complexed to a polycation.

The preparation of the lipopolyplex of the invention can be carried out by different methods well known to those skilled in the art. For example, the lipopolyplex of the invention can be obtained by mixing a complexed nucleic acid (cationic polymer) with a preparation of liposomes according to the invention.

According to an embodiment, the lipopolyplex of the invention is obtained by mixing:
a nucleic acid, preferably an mRNA, encoding a tumour antigen, the nucleic acid being complexed to the partially histidinylated polylysine and comprising one molecule of PEG 5 kDa (PEG-HpK); and
a preparation of liposomes according to the invention.

According to an embodiment, the lipopolyplex of the invention is obtained by mixing:
a nucleic acid, preferably an mRNA, encoding a tumour antigen, the nucleic acid being complexed to the partially histidinylated polylysine and comprising one molecule of PEG 5 kDa (PEG-HpK); and
a preparation of liposomes comprising 5% of the compound according to the invention, 47.5% of lipid KLN25, and 47.5% of lipid MM27, in mole percent with respect to the total number of moles of lipids.

According to an embodiment, the liposomes, lipoplexes or lipopolyplexes of the invention are dispersed in an aqueous solution. In a particular embodiment, the liposomes, lipoplexes or lipopolyplexes of the invention are dispersed in physiological serum or in PBS.

According to an embodiment, the invention also relates to an aqueous solution or a dispersion comprising the liposomes, lipoplexes or lipopolyplexes of the invention.

According to another embodiment, the present invention also relates to an emulsion comprising the liposomes, lipoplexes or lipopolyplexes of the invention.

In an embodiment, the emulsion comprising the liposomes, lipoplexes or lipopolyplexes of the invention is an oil-in-water emulsion, a water-in-oil emulsion or a water-in-oil-in-water emulsion.

According to an embodiment, the present invention also relates to a pharmaceutical composition comprising the liposomes, lipoplexes or lipopolyplexes of the invention and at least one pharmaceutically acceptable excipient. Examples of pharmaceutically acceptable excipients include, but are not limited to, water, saline water, dextrose, glycerol, and others, or combinations of these excipients. According to an embodiment, the pharmaceutical composition may also comprise humidifiers, emulsifiers, buffers, adjuvants, and others.

In an embodiment, the pharmaceutical composition further comprises at least one immune checkpoint inhibitor (ICI). Many tumours manage to express molecules that inhibit the immune response and thus to escape the control of the immune system. Immune checkpoint inhibitors are molecules that act against such inhibitor mechanisms developed by the tumour cells. Examples of immune checkpoint inhibitor include, without being limited to, CTLA-4 inhibitors (for example ipilumab), PD-1 inhibitors (for example pembrolizumab and nivolumab) and PD-L1 inhibitors (for example atezolizumab, avelumab and durvalumab).

According to an embodiment, the pharmaceutical composition of the invention can be administered to the subject by any suitable method of administration. Thus, according to an embodiment, the pharmaceutical composition of the invention can be formulated for oral administration, topical administration or an injection. According to an embodiment, the pharmaceutical composition of the invention can also be formulated for systemic, intravenous, intraperitoneal, intramuscular, intranodal, intracoronary, intra-arterially, subcutaneous, intradermal, transdermal, intratumoral, intraocular, pulmonary, by inhalation, direct injection into a tissue, or par electroporation or sonoporation administration. In a preferred embodiment, the pharmaceutical composition of the invention is formulated to be administered by injection, preferably by intradermal or intravenous injection, more preferably by intradermal injection.

In an embodiment, the pharmaceutical composition according to the invention is formulated for oral administration. Examples of forms suitable for oral administration include, but are not limited to, tablets (including extended-release tablet), capsules, powders, granules, pills (including sugar-coated pills), gelcaps (including flexible gelatin capsules), oral suspensions, drinkable solutions, and other similar forms.

In another embodiment, the pharmaceutical composition of the present invention is formulated to be injected. Examples of forms suitable for administration by injections include, but are not limited to, sterile aqueous solution, dispersions, emulsions, suspensions, solid forms suitable for the preparation of solutions or suspensions by adding a liquid before use such as, for example, powders.

In another embodiment, the pharmaceutical composition of the present invention is formulated for a topical application. Examples of forms suitable for administration by injections include, but are not limited to milks, creams, balms, oils, lotions, gels, salves, sprays or drops, such as eye drops.

According to an embodiment, the invention also relates to a vaccine adjuvant comprising the liposomes, lipoplexes or lipopolyplexes of the invention and at least one pharmaceutically acceptable excipient.

According to an embodiment, the invention also relates to a vaccine comprising the liposomes, lipoplexes or lipopolyplexes of the invention themselves comprising at least one immunogenic molecule, and at least one pharmaceutically acceptable excipient. In an embodiment, the vaccine of the invention can furthermore comprise at least one additional adjuvant. In another embodiment, the vaccine further comprises at least one immune checkpoint inhibitor.

In an embodiment, the vaccine of the invention can be formulated under different forms, including for example a solution, a dispersion or an emulsion.

In the embodiment according to which the vaccine of the invention is formulated under the form of an emulsion, the vaccine preferentially comprises one or several surfactant(s).

According to an embodiment and in order to improve the preservation thereof, the vaccine of the invention may be lyophilised. The vaccine of the invention can thus have a lyophilised form. In this embodiment, the vaccine comprises one or several agents assisting in lyophilisation. Agents assisting in lyophilisation are well known to those skilled in the art. These agents assisting in lyophilisation include in particular sugars such as lactose and mannitol.

In an embodiment, the vaccine of the invention can comprise one or several stabilising agents, for example in order to prolong the duration of the preservation of the vaccine or in order to improve the effectiveness of the lyophilisation. Examples of stabilising agents include, without being limited to, SPGA (Sucrose-Phosphate-Glutamate-Albumin), sugars such as sorbitol, mannitol, trehalose, starch, sucrose, dextran or glucose, proteins such as albumin or casein or the degradation products thereof, mixtures of amino acids such as lysine or glycine, and buffers such as alkali metal phosphates.

In an embodiment, the vaccine of the invention is administered to the subject by one of the conventional methods of vaccination including an injection, for example intradermal, intramuscular, intraperitoneal, or subcutaneous; a topical administration, for example a transdermal application or an application by intranasal spray; or an oral administration. The vaccine of the invention can be administered in a single dose or in several doses.

In the embodiment according to which the vaccine is injected, the formulation to be injected may have the form of a solution or sterile dispersion, or of a sterile lyophilised powder allowing for the extemporaneous preparation of a solution or sterile dispersion that can be injected. In order to prevent any contamination by microorganisms, one or several preservatives can be added to the vaccine such as for example antibacterial and antifungal agents such as parabens, chlorobutanol, phenol, sorbic acid, thiomersal and other similar agents. It may also be preferable to add to the vaccine one or several isotonic agents such as sugars or sodium chloride in order to reduce the pain caused by the injection. In order to prolong the absorption of an injected vaccine of the invention, one or several absorption-retarding agents can be added to the vaccine such as for example aluminium monostearate or gelatin.

It is understood that the total daily use of the liposome, lipoplex, lipopolyplex, pharmaceutical composition or vaccine of the invention will be adjusted by the attending physician in the framework of his medical opinion. The therapeutically effective dose specific to each patient will depend on a variety of factors including the disorder treated and the severity thereof; the activity of the compound used; the specific composition used; the age, weight, general health, gender and diet of the patient, the duration and the mode of administration; the duration of the treatment; the drugs used in combination or concomitantly with the compound used, and other similar factors known in the medical field.

According to an embodiment, the liposome, lipoplex, lipopolyplex, the pharmaceutical composition or the vaccine of the invention is administered once, twice, 3 times, 4 times, 5 times, 6 times, 7 times, 8 times, 9 times, or 10 times to the subject.

In an embodiment, the liposome, lipoplex, lipopolyplex, the pharmaceutical composition or the vaccine of the invention is administered to the subject at least once a day. Preferably, the liposome, lipoplex, lipopolyplex, the pharmaceutical composition or the vaccine of the invention is administered once a day. In another embodiment, the liposome, lipoplex, lipopolyplex, the pharmaceutical composition or the vaccine of the invention is administered to the subject at least once a week. For example, the liposome, lipoplex, lipopolyplex, the pharmaceutical composition or the vaccine of the invention can be administered once a week, twice, three times, four times or up to seven times per week.

In an embodiment, the liposome, lipoplex, lipopolyplex, the pharmaceutical composition or the vaccine of the invention is administered to the subject before the symptoms appear, i.e. the liposome, lipoplex, lipopolyplex, the pharmaceutical composition or the vaccine of the invention is administered prophylactically.

In an embodiment, the liposome, lipoplex, lipopolyplex, the pharmaceutical composition or the vaccine of the invention is administered to the subject after the symptoms have appeared, i.e. the liposome, lipoplex, lipopolyplex, the pharmaceutical composition or the vaccine of the invention is administered therapeutically.

In an embodiment, the liposome, lipoplex, lipopolyplex, the pharmaceutical composition or the vaccine of the invention is administered according to an optimal administration protocol. According to the invention, an optimal administration protocol comprises suitable dosage parameters and modes of administration that lead to the stimulation or to the triggering of an immune response in the subject.

The effective dosage parameters can be determined by conventional methods for a disease in particular. Examples of such methods include, but are not limited to survival rates, side effects, the progression or regression of the disease.

In particular, the effectiveness of the dose of liposomes, lipoplexes, lipopolyplexes, pharmaceutical composition or vaccine according to the invention for treating cancer can be determined by evaluating the response rate, which corresponds to the proportion of patients for whom the tumour regresses or does not grow under treatment.

The present invention also relates to a liposome, lipoplex or lipopolyplex according to the invention for its use for vectorisation, i.e. the transmembrane transfer of a molecule of interest. The present invention therefore also relates to the use of the liposome, lipoplex or lipopolyplex according to the invention for the vectorisation, i.e. the membrane transfer of a molecule of interest.

According to an embodiment the present invention relates to a liposome, lipoplex or lipopolyplex according to the invention for its use for the transfection of cells, preferably for the in vivo transfection of cells. According to another embodiment the present invention relates to the use of a liposome, lipoplex or lipopolyplex according to the invention for the in vitro or ex vivo transfection of cells.

The present invention therefore relates to a method of in vitro or ex vivo transfection of cells, comprising the use of a lipoplex or lipopolyplex according to the invention.

According to an embodiment, the present invention relates to a liposome, lipoplex or lipopolyplex according to the invention for its use for the delivery of prodrugs, sensitisers or visualising agent.

According to an embodiment, the present invention relates to a liposome, lipoplex or lipopolyplex according to the invention for its use for gene therapy, topical treatments (dermatology, ophthalmology) or bactericidal activities.

The invention further relates to a cosmetic composition comprising a liposome, lipoplex or lipopolyplex according to the invention. Another object of the invention is also the cosmetic use of a liposome, lipoplex or lipopolyplex according to the invention.

The invention also relates to a liposome, lipoplex or lipopolyplex according to the invention for its use for targeted vectorisation. The present invention therefore also relates to the use of the liposome, lipoplex or lipopolyplex according to the invention for targeted vectorisation. According to an embodiment, the compound of the invention contained in the liposome, lipoplex or lipopolyplex of the invention is recognised and bound by a particular type of cells.

In an embodiment, the compound of the invention contained in the liposome, lipoplex or lipopolyplex of the invention is recognised and bound by the cells expressing a lectin on their surface. In another embodiment, the compound of the invention contained in the liposome, lipoplex or lipopolyplex of the invention is recognised and bound by the cells expressing a type C lectin on their surface. In another embodiment, the compound of the invention contained in the liposome, lipoplex or lipopolyplex of the invention is recognised and bound by the cells expressing on their surface the lectins DC-SIGN (CD209), DEC205/CD205, langerin (CD207), CLEC9A, CLEC4D, CD40, and/or the mannose receptor (MR).

In an embodiment, the compound of the invention contained in the liposome, lipoplex or lipopolyplex of the invention is recognised and bound by antigen-presenting cells. In another embodiment, the compound of the invention contained in the liposome, lipoplex or lipopolyplex of the invention is recognised and bound by macrophages, dendritic cells and/or Langerhans cells. In a preferred embodiment, the compound of the invention contained in the liposome, lipoplex or lipopolyplex of the invention is recognised and bound by dendritic cells.

According to an embodiment the present invention relates to a liposome, lipoplex or lipopolyplex according to the invention for its use for the transfection of antigen-presenting cells, preferably for the transfection of macrophage, dendritic cells and/or Langerhans cells, more preferably for the transfection of dendritic cells.

The invention also relates to a compound of the invention, a liposome, lipoplex or lipopolyplex according to the invention for its use as an adjuvant.

In an embodiment, the compound of the invention, the liposome, lipoplex or lipopolyplex of the invention has an in vitro adjuvant effect. In an embodiment, the compound of the invention, the liposome, lipoplex or lipopolyplex of the invention has an in vivo adjuvant effect.

The invention also relates to a compound of the invention, a liposome, lipoplex or lipopolyplex according to the invention for its use as a vaccine adjuvant.

In an embodiment, the liposome, lipoplex or lipopolyplex of the invention administered to a subject induces an immune response in the subject. In an embodiment, the liposome, lipoplex or lipopolyplex of the invention administered to a subject induces an immune response in the subject that does not depend on the presence in the liposome, lipoplex or lipopolyplex of the invention of an immunogenic molecule of interest.

The invention also relates to a liposome, lipoplex or lipopolyplex according to the invention for its use for vaccination. The invention also relates to a liposome, lipoplex or lipopolyplex according to the invention comprising at least one molecule of interest capable of inducing an immune response for its use for vaccination. The invention also relates to a liposome, lipoplex or lipopolyplex according to the invention comprising at least one molecule of interest capable of inducing an immune response for its use as a vaccine.

In an embodiment, the liposome, lipoplex or lipopolyplex of the invention is used for a preventive vaccination. In another embodiment, the liposome, lipoplex or lipopolyplex of the invention is used for a therapeutic vaccination.

In an embodiment, the liposome, lipoplex or lipopolyplex of the invention is used as a cancer vaccine. In a preferred embodiment, the liposome, lipoplex or lipopolyplex of the invention is used for a therapeutic vaccination in the treatment of cancer.

In an embodiment, the term “cancer” or “tumour” includes all of the proliferative diseases. In an embodiment, proliferative diseases include neoplasms, dysplasia, premalignant or precancerous lesions, abnormal growths of cells, malignant tumours, and cancers or metastases. In an embodiment, the cancer is selected from the group comprising leukaemia, non-small cell lung cancer, small cell lung cancer, central nervous system cancer (CNS), melanoma, ovarian cancer, kidney cancer, prostate cancer, breast cancer, glioma, colon cancer, bladder cancer, sarcoma, pancreatic cancer, colorectal cancer, head and neck cancer, liver cancer, bone cancer, spinal cord cancer, stomach cancer, intestinal cancer, cancer of the oesophagus, thyroid cancer, hematologic cancer, and lymphoma.

In an embodiment, the cancer is a breast cancer, melanoma or hepatocellular carcinoma. In an embodiment, the breast cancer is a triple negative breast cancer, i.e. a cancer of the breast characterised by the absence of expression of the receptors to oestrogens, receptors to progesterone and HER2 (human epidermal growth factor receptor 2) by the cancer cells.

In an embodiment, the liposome, lipoplex or lipopolyplex of the invention used as a cancer vaccine comprises at least one tumour antigen or at least one nucleic acid encoding a tumour antigen. In an embodiment, the liposome, lipoplex or lipopolyplex of the invention used for a therapeutic cancer vaccination comprises at least one tumour antigen or at least one nucleic acid encoding a tumour antigen.

In an embodiment, the liposome, lipoplex or lipopolyplex of the invention used for a therapeutic cancer vaccination comprises at least one tumour antigen or at least one nucleic acid encoding a tumour antigen chosen from the non-exhaustive list comprising: CDK4 (cyclin-dependent kinase 4), p1S^{1″1′4′3}, AFP, β-catenin, caspase 8, p53, mutated version of p21^{Ras}, Bcr-abl chimera, MUM-I MUM-2, MUM-3, ELF2M, HSP70-2M, HST-2, KIAA0205, RAGE, myosin/m, 707-AP, CDC27/m, ETV6/AML, TEL/Amll, Dekcain, LDLR/FUT, Pml-RARαTEL/AMLI, NY-ESO-I, members of the MAGE family (Melanoma-associated antigen) MAGE-Al, MAGE-A2, MAGE-A3, MAGE-A4, MAGE-A6, MAGE-10, MAGE-12, BAGE, DAM-6, DAM-10, members of the GAGE family (G antigen) GAGE-1, GAGE-2, GAGE-3, GAGE-4, GAGE-5, GAGE-6, GAGE-7B, GAGE-8, NA-88A, CAG-3, RCC-associated antigen G250, oncoproteins E6 and E7 derived from HPV (human papilloma virus), Epstein Barr virus antigens EBNA2-6, LMP-I, LMP-2, gp77, gp100, MART-1/Melan-A, tyrosinase, TRP-I and TRP-2 (tyrosinase-related protein), PSA, PSM, MC1R, ART4, CAMEL, CEA, CypB, HER2/neu, hTERT, hTRT, iCE, Muc1, Muc2, PRAME, RU1, RU2, SART-1, SART-2, SART-3, and WT1. In an embodiment, the liposome, lipoplex or lipopolyplex of the invention used for therapeutic cancer vaccination comprises at least one tumour antigen or at least one nucleic acid encoding a tumour antigen chosen from the non-exhaustive list comprising: CAP-1, CD 4/m, cell surface proteins of the claudin family CLAUDIN-6, CLAUDIN-18.2 and CLAUDIN-12, c-myc, CT, GnT-V, HAGE, HAST-2, LAGE, NF1, NY-BR-1, proteinase 3, SAGE, SCGB3A2, SCP1, SCP2, SCP3, SSX, SURVrVin, TPI/m, TPTE, CDK4 (cyclin-dependent kinase 4), plS^{1″1′4′3}, AFP, β-catenin, caspase 8, p53, mutated version of p21^{Ras}, Bcr-abl chimera, MUM-I MUM-2, MUM-3, ELF2M, HSP70-2M, HST-2, KIAA0205, RAGE, myosin/m, 707-AP, CDC27/m, ETV6/AML, TEL/Amll, Dekcain, LDLR/FUT, Pml-RARαTEL/AMLI, NY-ESO-I, members of the MAGE family (Melanoma-associated antigen) MAGE-Al, MAGE-A2, MAGE-A3, MAGE-A4, MAGE-A5, MAGE-A6, MAGE-A7, MAGE-A8, MAGE-A9, MAGE-A1O, MAGE-A11, MAGE-A12, MAGE-B, MAGE-C, BAGE, DAM-6, DAM-10, members of the GAGE family (G antigen) GAGE-1, GAGE-2, GAGE-3, GAGE-4, GAGE-5, GAGE-6, GAGE-7B, GAGE-8, NA-88A, CAG-3, RCC-associated antigen G250, oncoproteins E6 and E7 derived from HPV (human papilloma virus), Epstein Barr virus antigens EBNA2-6, LMP-I, LMP-2, gp77, gp100, MART-1/Melan-A, tyrosinase, TRP-I and TRP-2 (tyrosinase-related protein), TRP-2-INT2, PSA, PSM, MC1R, ART4, CAMEL, CEA, CypB, HER2/neu, hTERT, hTRT, iCE, Muc1, Muc2, PRAME, RU1, RU2, SART-1, SART-2, SART-3, WT and WT1.

According to an embodiment, the liposome, lipoplex or lipopolyplex of the invention used for a therapeutic cancer vaccination comprises at least one tumour antigen or at least one nucleic acid encoding a tumour antigen chosen from the list comprising oncoproteins E6 and E7 derived from HPV (human papilloma virus), MART-1/Melan-A. According to another embodiment, the liposome, lipoplex or lipopolyplex of the invention used for a therapeutic cancer vaccination comprises at least oncoprotein E7 derived from HPV (human papilloma virus) or at least one nucleic acid encoding oncoprotein E7 or a peptide of oncoprotein E7.

In an embodiment, the subject is suceptible to or suspected of suffering from a disease, preferably an infectious disease or a cancer.

Examples of risks of developing cancer include, but are not limited to, a family history or genetic predisposition, age, consumption of alcohol, tobacco, exposure to toxic and/or carcinogenic substances, exposure to radiation, exposure to the sun, chronic inflammation, diet, and others.

Examples of risks of developing infectious diseases include, but are not limited to, exposure to bacteria, viruses, mushrooms, parasites, and others.

In an embodiment, the term “infectious diseases” includes all diseases caused by an infectious agent, such as a bacterium, a virus, a mushroom or other and diseases caused by a parasite.

In an embodiment, the infectious disease is selected from the flu, tuberculosis, pneumonias of bacterial origin, pneumonias of viral origin, Lyme disease, human immunodeficiency virus (HIV) infections, and acquired immunodeficiency syndrome (AIDS).

Examples of diseases caused by a virus include, but are not limited to human immunodeficiency virus (HIV) infections, acquired immunodeficiency syndrome (AIDS), adenovirus infections, alpha-virus infections, arbovirus infections, pneumonias of viral origin, Bell’s paralysis, Borna disease, infections with a virus of the Bunyaviridae family, infections with a virus of the Caliciviridae family, chicken pox, cold, condyloma accuminata, infections with a coronavirus, infections with coxsackievirus, infections with a cytomegalovirus, Chikungunya, dengue, infections wtih a DNA virus, infections with an RNA virus, contagious ecthyma, encephalitis, encephalitis caused by an arbovirus, encephalitis caused by herpes simplex virus, infections caused by the Epstein-Barr virus, infectious erythema, exanthema subitum (or roseola infantum), chronic fatigue syndrome, infections with a hantavirus, viral haemorrhagic fever, Ebola virus disease, Marburg virus disease, (viral hepatitis or infections with hepatitis A (HAV) virus, with hepatitis B (HBV) virus, with hepatitis C (HCV) virus or with hepatitis D (HDV) virus, herpes labialis, herpes simplex, herpes zoster (zona), infectious mononucleosis, flu, Lassa fever, measles, viral meningitis, molluscum contagiosum, smallpox, mumps, myelitis, infections with a papillomavirus, infections with a human papillomavirus (HPV), infections with a virus of the Paramyxoviridae family, sandfly fever, poliomyelitis, infections with a polyomavirus, postpoliomyelitis syndrome, rabies, respiratory infections due to respiratory syncytial virus (RSV), rubella, severe acute respiratory syndrome (SARS), subacute sclerosing panencephalitis, tick-borne diseases, warts, West Nile fever, viral diseases, infections caused by a virus of the genus Flavivirus, yellow fever, zika, zoonotic diseases, and others.

In an embodiment, the infectious disease is a disease caused by a virus and selected from acquired immunodeficiency syndrome (AIDS) or infection with human immunodeficiency virus (HIV), viral hepatitis or infections with hepatitis A (HAV) virus, with hepatitis B (HBV) virus, with hepatitis C (HCV) virus or with hepatitis D (HDV) virus, and infections with a human papillomavirus (HPV).

Examples of diseases caused by a bacterium or a mushroom include, but are not limited to abscess, actinomycosis, anaplasmosis, anthrax, reactive arthritis,aspergillosis, bacteraemia, bacterial infections and mycoses, infections with the Bartonella bacterium, botulism, brain abscess, brucellosis (or Malta fever), infections with a bacterium of the Burkholderia family, infections with a bacterium of the genus Campylobacter, candidiasis such as the candidiasis caused by Candida albicans, vulvovaginal candidiasis, cat-scratch disease (or benign inoculation lymphoreticulosis, or benign lymphogranuloma), cellulitis, infections of the central nervous system, canker, chlamydia infections, cholera, Clostridium infections, coccidioidomycoses such as coccidioidomycosis caused by Coccidioides immitis, corneal ulcer, cross-infections, blastomycosis, cryptococcosis, dermatomycoses, diphtheria, ehrlichiosis, pleural empyem, bacterial endocarditis, endophthalmitis, staphylococcal enterocolitis, erysipelas, infections caused by Escherichia coli, necrotizing fasciitis, Fournier gangrene, gas gangrene, furunculosis, infections with a bacterium of the genus Fusobacterium, gonorrhoea, infections wtih a gram-negative bacterium, infections with a gram-positive bacterium, granuloma inguinal, hidradenitis suppurativa, histoplasmosis, hordeolum (or stye), impetigo, infections with a bacterium of the genus Klebsiella, legionellosis (Legionnaires’ disease), leprosy, leptospirosis, Listeria infections, Ludwig’s angina, lung abscess, Lyme disease, lymphogranuloma venereum, eumycetoma, melioidosis, bacterial meningitis, Mycobacterium infections, infections by mycoplasma, mycoses, infections by Nocardia, onychomycosis, osteomyelitis, paronychia, pelvic inflammatory disease, plague, pneumonias of bacterial origin, pneumococcal infections, Pseudomona infections, psittacosi, puerperal infection, rat-bite fever, relapsing fever, respiratory tract infections, retropharyngeal abscess, rheumatic fever, rhinoscleroma, rickettsial infections, Rocky Mountain Spotted fever, Q fever, salmonella, scarlet fever, typhus, epidemic typhus, septicaemia, bacterial sexually transmitted infections, septic shock, bacterial skin diseases, infectious skin diseases, staphylococcal infections, streptococcal infections, syphilis, tetanus, tick-borne diseases, ringworm, trachoma, tuberculosis, tularaemia, typhoid fever, urinary tract infections, gastric ulcer linked to an infection by Helicobacter pylori, Whipple’s disease, whooping cough, infections caused by Vibrio, yaws, infections by Yersinia, zoonotic disease, mucormycosis or zygomycosis, and others.

Examples of diseases caused by a parasite include, but are not limited to malaria, sleeping sickness, leishmaniasi, toxoplasmosis, and others.

In another embodiment, the subject suffers from a disease, preferably an infectious disease or a cancer.

In an embodiment, the subject is an animal, preferably a mammal, more preferably a human. In an embodiment, the subject is a man. In another embodiment, the subject is a woman. In an embodiment, the subject is a child.

An object of the present invention is also a method for vectoring, i.e. transferring a molecule of interest through a membrane, comprising the use of a liposome, lipoplex or lipopolyplex according to the invention.

The present invention also relates to a method for inducing an immune response in a subject comprising the administration of the liposome, lipoplex or lipopolyplex according to the invention.

The present invention also relates to a method for vaccinating a subject comprising the administration of the liposome, lipoplex or lipopolyplex according to the invention, preferably a liposome, lipoplex or lipopolyplex comprising at least one molecule of interest capable of inducing an immune response.

According to an embodiment, the vaccination is preventive. According to another embodiment, the vaccination is therapeutic.

In an embodiment, the method for vaccinating a subject according to the invention is a method for vaccination against cancer, preferably a method of therapeutic vaccination against cancer.

Another object of the present invention relates to a method for preventing cancer in a subject comprising the administration of the liposome, lipoplex or lipopolyplex according to the invention, preferably a liposome, lipoplex or lipopolyplex comprising at least one tumour antigen or a nucleic acid encoding a tumour antigen.

The present invention invention also relates to a method for treating cancer in a subject comprising the administration of the liposome, lipoplex or lipopolyplex according to the invention, preferably a liposome, lipoplex or lipopolyplex comprising at least one tumour antigen or a nucleic acid encoding a tumour antigen.

### BRIEF DESCRIPTION OF THE DRAWINGS

FIG. 1 is a set of graphs showing the binding of the bare lipopolyplexes LPR, LPR-MN or LPR-triMN to cells expressing lectin receptors: (A) cellules 293T expressing or not the receptor DC-SIGN; (B) human monocyte-derived dendritic cells (MoDCs); (C) human blood mononuclear cells (PBMCs); (D) human dendritic cells isolated from the blood (panDCs).

FIG. 2 is a set of graphs showing the binding of the lipopolyplexes LPR-MN, diether LPR-MN or LPR-triMN to cells expressing lectin receptors: (A) murine dendritic cells DC 2.4; (B) murine spleen cells; (C) human monocyte-derived dendritic cells (MoDCs).

FIG. 3A is a set of graphs showing the expression of the activation marker CD80 by MoDCs FITC- or FITC+ dendritic cells after incubation in the presence of increasing concentrations of lipopolyplexes LPR-MN or LPR-triMN. The FITC- cells did not capture the lipopolyplexes, the FITC+ cells captured the lipopolyplexes.

FIG. 3B is a histogram showing the expression of the activation markers HLA-DR and CD83 by MoDCs cells that have been incubated in the presence of 2.5 µg/ml of LPR-MN or LPR-triMN.

FIG. 4A is a graph showing the expression of the GFP by dendritic cells MoDCs incubated at t0 with lipopolyplexes LPR-MN containing mRNA of the GFP then at t6h with LPS (positive control) or LPR-MN or LPR-triMN liposomes containing RNA control single strand PolyU (ssPolyU).

FIG. 4B is a graph showing the expression of the activation marker CD80 by dendritic cells MoDCs incubated at t0 with LPR-MN containing mRNA of the GFP then at t6h with LPS (positive control) or LPR-MN or LPR-triMN containing RNA control single strand PolyU (ssPolyU).

FIG. 4C is a histogram showing the expression of the activation markers CD80, CD83 and HLA-DR by dendritic cells MoDCs incubated in a first step with LPR-MN containing mRNA of the GFP for 6 h then in a second step with LPS (positive control) or LPR-MN or LPR-triMN containing RNA control single strand PolyU (ssPolyU) for 12 h hours.

FIG. 5A is a set of photographs showing the two injection sites (indicated by arrows) on the back of mice having received 24 h (1) and 48 h (2) beforehand an injection of PBS, of LPR-MN lipopolyplexes or of LPR-triMN lipopolyplexes.

FIG. 5B is a set of photographs showing the microscopic analysis after haematoxylin-eosin marking of a section of 10 µm of skin on the injection site (point 48 h) and of the inguinal lymph node draining the injection sites of mice having received 24 h (1) and 48 h (2) beforehand an injection of PBS, of LPR-MN or of LPR-triMN.

FIG. 6 is a set of photographs showing the analysis via fluorescence microscopy of popliteal lymph nodes taken from mice having received 6 h beforehand an injection of PBS, of LPR-MN or of LPR-triMN marked with rhodamine. The marker CD169 (anti-CD169-APC) makes it possible to view the resident macrophages of the lymph node subcapsular sinus. The LPR can be viewed thanks to a marking with rhodamine. The rhodamine signal co-located with the signal CD169 is indicated by asterisks (*). The rhodamine signal located on the cells with extended branches that do not express CD169 (probably dendritic cells) is indicated by arrow.

FIG. 7 is a set of graphs showing: (A) the absolute value (number) and (B) the relative percentage of dendritic cells; and within these total dendritic cells, (C) the percentage of activated dendritic cells (Ly6C+ cells) and (D) of inflammatory dendritic cells (LY6G+ cells) present in the lymph nodes draining the injection site taken from mice having received 24 h beforehand an injection of PBS, of bare LPR, of bare diether LPR, of LPR-MN, or of LPR-triMN.

FIG. 8 is a set of histograms showing the percentage of CD4+ (A) and CD8+ (B) lymphocytes expressing interferon-y after a sensitisation by dendritic cells having incorporated the indicated lipopolyplexes containing mRNA of oncoprotein E7 (bare LPR, LPR-MN or LPR-triMN) followed by a stimulation by dendritic cells loaded beforehand with peptides E7. As a control, the mRNA is replaced with an RNA single strand PolyU (ssPolyU).

FIG. 9 is a set of graphs showing: (A) the secretion of interferon-y by isolated lymphocytes of the spleen of mice vaccinated with LPR-triMN-E7/E7-DC-LAMP administered by different paths (IV: intravenous, SC: subcutaneous, ID: intradermal); (B) the secretion of interferon-y by isolated lymphocytes of the spleen of mice vaccinated at day 0 and day 2 with an injection of PBS, or of different LPR (bare LPR, bare diether LPR, LPR-MN, LPR-triMN) containing mRNA of oncoprotein E7.

FIG. 10A is a set of graphs showing the volume of the tumour developed by mice which were injected with 50,000 cells of the syngeneic tumoural line TC-1 and which were then vaccinated with PBS, LPR-MN-ssPolyU, LPR-MN-E7/E7-DC-LAMP, LPR-triMN-ssPolyU or LPR-triMN-E7/E7-DC-LAMP. Each curve corresponds to the volume of the tumour developed by one mouse. The arrows indicated the days on which the mice were vaccinated (D7 and D9).

FIG. 10B is a graph showing the survival rate of these mice.

FIG. 11 is a set of graphs showing the volume of the tumour developed by mice which were injected with B16F0 tumour cells expressing MART1 (A) or EG7 tumour cells expressing OVA (B) and which were then vaccinated with PBS, LPR-triMN-ssPolyU or LPR-triMN containing mRNA of MART1 or OVA, respectively. NS: no statistically significant difference (i.e. p > 0.05), * p < 0.05, ** p < 0.01.

### EXAMPLES

This invention shall be understood better when reading the following examples that non-limitingly illustrate the invention.

### Example 1: Synthesis of a Tri-mannosylated Lipid of the Invention

### Material and Methods

### Nuclear Magnetic Resonance (NMR)

Fourier-transform spectrometers BRUKER ARX 400 and BRUKER Avance 400 (400.13 MHz for the proton, 100.61 MHz for the carbon, ENSCR).

The chemical shifts are expressed in parts per million (ppm) with respect to the chemical shift of the deuterated solvent used as a reference. The multiplicity of signals is shown by using the following abbreviations: s, singlet; d, doublet; t, triplet; q, quadruplet; m, multiplet or massive that cannot be analysed. The coupling constants (J) are expressed in Hertz (Hz). The spectrometric data 13C is determined using fully decoupled spectra and heteronuclear 2D correlated spectra.

### Mass Spectrometry

High resolution mass spectrometry MS/MS ZabSpec TOF Micromass (Centre Regional de Mesures Physiques de l′Ouest, Université de Rennes 1).

The ionisation mode used is positive electrospray (ESI+-MS). The ion acceleration voltage is 4 kV and the temperature of the source is equal to 60° C. (mode of introduction by infusion). The compounds are dissolved beforehand in methanol or in dimethylsulfoxide.

### Abbreviations

AcOEt: ethyl acetate;
DIEA: N,N′-diisopropylethylamine;
Eb: boiling temperature;
EP: petroleum ether;
And: ethyl;
EtOH: ethanol;
HRMS: high resolution mass spectrometry;
Me: methyl;
MeOH: methanol;
ppm: parts per million;
NMR: nuclear magnetic resonance;
TBTU: tetrafluoroborate of O-(benzotriazol-1-yl)-1,1,3,3-tetramethyluronium;
TEMPO: 2,2,6,6-tetramethylpiperidine-1-oxyl;
THF: tetrahydrofurane.

### Synthesis of Carboxylic Acid Tri-Mannosylated Ligand (Comprising an Oligo(Propylene Glycol)N Spacer Where N = 2)

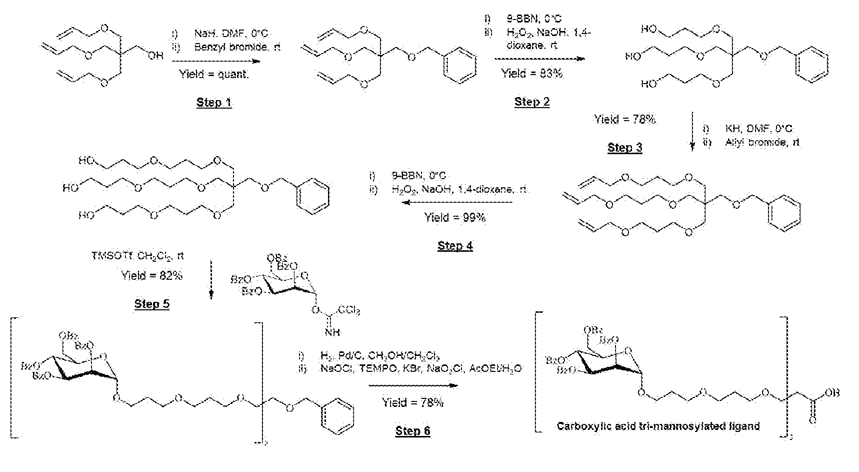

### Step 1: Benzylation of the Pentaerythritol Triallyl Ether

To a suspension of sodium hydride NaH (2.6 g, 90 mmol) in anhydrous DMF (100 mL), is added drop-by-drop the commercial pentaerythritol allyl ether (7.70 g; 300 mmol) purified beforehand by chromatography column on silica gel (eluent cyclohexane/ethyl acetate: 9/1, v/v). The medium is stirred 2 hours at 0° C. then benzyl bromide (7.7 g, 450 mmol) is added drop-by-drop at this temperature. The medium is left under stirring at ambient temperature for 17 hours then MeOH (6 mL) is added drop-by-drop at 0° C. The solvents are evaporated in the vacuum rotary evaporator then the vane pump. The yellowish residue is taken up in dichloromethane then is washed with water and by an aqueous solution saturated with NaCl. The organic phase is dried on MgSO4 and vacuum concentrated. The crude product is purified by chromatography on a column of silica gel (eluent: EP/AcOEt): 9/1, v/v) in order to provide the benzyl derivative (10.45 g) in the form of a colourless oil with a quantitative yield.

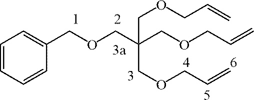

NMR ¹H (CDCl3, 400 MHz) δ (ppm):

3.50 (6H, s, H3), 3.56 (2H, s, H2), 3.95 (6H, dt, J = 5.3, 1.5 Hz, H4), 4.65 (2H, s, H1), 5.12 (3H, dq, J = 10.4, 1.7 Hz, H6a), 5.24 (3H, dq, J = 17.2, 1.7 Hz, H6b), 5.87 (3H, m, H5), 7.24-7.35 (7H, m, Har).

### Step 2: Hydroboration-oxidation

The triallyl compound (10 g, 28.8 mmol) is dissolved in 50 mL of anhydrous dioxane. The 9-BBN (0.5 M in THF) (519 mL, 0.259 mol) is added to the reaction medium cooled beforehand to 0° C. and the mixture is stirred for 24 hours at ambient temperature. The sodium hydroxide 3 M (577 mL, 75 eq.) and the hydrogen peroxide 10 M (115 mL 1.15 mol) are added at 0° C. to the reaction medium and the mixture is stirred 12 hours at ambient temperature. The mixture is extracted 3 times with AcOEt then the organic phase is dried on MgSO4, filtered, concentrated under reduced pressure and purified by chromatography on silica gel (eluent AcOEt/EP/MeOH: 10/5/1) in order to give 9.57 g of triol in the form of a colourless oil with a yield of 83%.

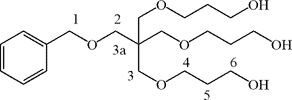

NMR ¹H (CDCl3, 400 MHz) δ (ppm):

1.75 (6H, p, J = 5.5, 5.3 Hz, H5), 3.28 (3H, s.large, OH), 3.42 (6H, s, H3), 3.44 (2H, s, H2), 3.56 (6H, t, J = 5.5 Hz, H4), 3.70 (6H, t, J = 5.3 Hz, H6), 4.62 (2H, s, H1), 7.24-7.35 (7H, m, Har).

### Step 3: Allylation of the Triol

The triol (7.8 g, 19.4 mmol) is dissolved in 100 mL of anhydrous DMF and is added drop-by-drop to the reaction medium containing potassium hydride (3.9 g, 97 mmol) in the DMF cooled beforehand to 0° C. The mixture is stirred 10 minutes at 0° C. before introducing drop-by-drop the allyl bromide (8.35 mL, 97 mmol). The stirring is prolonged 24 hours at ambient temperature and the excess potassium hydride is hydrolysed with 100 mL of distilled water. The reaction medium is extracted 3 times with diethyl ether then the organic phase is washed with a saturated aqueous solution of NaCl, dried on MgSO4, filtered, concentrated under reduced pressure and purified by chromatography on silica gel (eluent EP/AcOEt: 85/15) in order to give 8.63 g of the triallyl product in the form of a colourless oil with a yield of 78%.

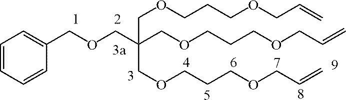

NMR ¹H (CDCl3, 400 MHz) δ (ppm):

1.81 (6H, q, J = 6.4 Hz, H5), 3.40 (6H, s, H3), 3.44-3.50 (14 H, m, H2, H4, H6), 3.95 (6H, ddd, J = 5.7, 1.5, H7), 4.48 (2H, s, H1), 5.15 (3H, ddd, J = 10.3, 3.3, 1.5 Hz, H9b), 5.26 (3H, ddd, J = 17.3, 3.5, 1.5 Hz, H9a), 5.90 (3H, ddt, J = 10.3, 6.8, 5.7 Hz, H8), 7.24-7.35 (7H, m, Har).

### Step 4: Hydroboration-oxidation

To a solution of the triallyl (7.9 g, 15 mmol) dissolved in 50 mL of anhydrous dioxane is added at 0° C. the 9-BBN (0.5 M in THF) (272 mL, 0.136 mol) and the mixture is stirred for 24 hours at ambient temperature. The sodium hydroxide 3 M (500 mL, 1.5 mol) and the aqueous hydrogen peroxide 35% (60 mL, 0.6 mol) are added at 0° C. to the reaction medium and the mixture is stirred 12 hours at ambient temperature. The mixture is extracted 3 times with AcOEt, dried on MgSO4, filtered, concentrated under reduced pressure and purified by chromatography on silica gel (eluent AcOEt/EP/MeOH: 10/5/1) in order to give 8.51 g of triol in the form of a colourless oil with a yield of 99%.

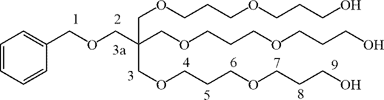 [SMILES: CCCCCCCC/C=C\CCCCCCCCOP(=O)(NCCn1cc[n+](C)c1)OCCCCCCCC/C=C\CCCCCCCC ; [I-]]

NMR ¹H (CDCl3, 400 MHz) δ (ppm):

1.79 (12 H, p, J = 5.7 Hz, H8, H5), 2.62 (3H, s.large, OH), 3.42 (6H, s, H3), 3.49-3.43 (14 H, m, H2, H4, H6), 3.55 (6H, t, J = 5.7 Hz, H7), 3.73 (6H, t, J = 5.4 Hz, H9), 4.64 (2H, s, H1), 7.24-7.35 (7H, m, Har).

### Step 5: Glycosylation of the Triol

To a solution of trichloroacetimidate (10 g, 13.4 mmol) and of triol (1.03 g, 1.79 mmol) solubilised in 125 mL of anhydrous CH₂Cl₂ is added the solution at 5% in the CH2Cl2 of TMSOTf (640 µL, 0.179 mmol) and the mixture is stirred 12 hours at ambient temperature. After having added 2 g of sodium bicarbonate to the reaction, the reaction medium is filtered, the precipitate is rinsed several times with CH₂Cl₂ and the filtrate is vacuum concentrated. The residue is purified by chromatography on silica gel (eluent EP/AcOEt: 6/4) in order to give 3.44 g of tri-mannosylated product in the form of a light yellow powder with a yield of 82%.

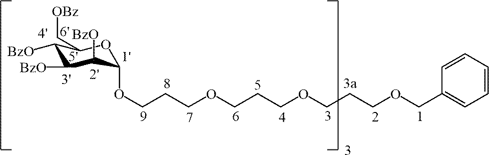 [SMILES: CCCCCCCC/C=C\CCCCCCCCOP(=O)(NCCc1c[nH]cn1)OCCCCCCCC/C=C\CCCCCCCC]

NMR ¹H (CDCl3, 400 MHz) δ (ppm):

1.77-1.84 (6H, m, J = 6.4 Hz, H5), 1.91-2.00 (6H, m, H8), 3.41-3.57 (26 H, m, H2, H3, H4, H6, H7), 3.57-3.68 (3H, m, H9b), 3.90-3.96 (3H, m, H9a), 4.40-4.50 (3H, m, H5′), 4.46-4.50 (5H, m, H1, H6b′), 5.08 (3H, d, J = 2.0 Hz, H1′), 5.70 (3H, dd, J = 3.2, 1.8 Hz, H2′), 5.91 (3H, dd, J = 10.1, 3.3 Hz, H3′), 6.11 (3H, t, J+10.1 Hz, H4′), 7.24-8.11 (65 H, m, Har).

### Step 6: Debenzylation - Oxidation

To a solution of benzyl ether (3.44 g, 1.49 mmol) dissolved in 30 mL of a mixture CH₂Cl₂/MeOH (4/1) is added at ambient temperature the Pd/C (340 mg, 10% by weight). The reaction medium is stirred under a hydrogen atmosphere one night at ambient temperature. The medium is diluted with CH₂Cl₂, filtered on celite then vacuum concentrated. The compound is then purified by chromatography on silica gel (eluent Cyclohexane/AcOEt: 6/4) in order to give 2.61 g of alcohol in the form of a white powder with a yield of 79%.

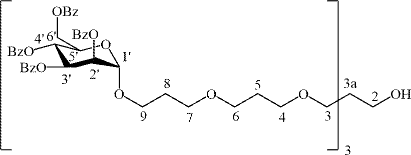 [SMILES: CCCCCCCCCCCCCCCCOCC(OCCC(C)CCCC(C)CCCC(C)CCCC(C)C)C(=O)O]

NMR ¹H (CDCl3, 400 MHz) δ (ppm):

1.81 (6H, p, J = 6.4 Hz, H5), 1.93 (6H, p, J = 6.4 Hz, H8), 3.04 (1H, t, J = 6.1 Hz, OH), 3.43 (6H, s, H3), 3.47 (6H, t, J = 6.3 Hz, H6), 3.50 (6H, t, J = 6.5 Hz, H4), 3.55 (6H, m, H7), 3.68 (5H, m, H9a, H2), 3.91 (3H, dt, J = 9.7, 6.4 Hz, H9b), 4.39-4.43 (3H, m, H5′), 4.47 (3H, dd, J = 12.0, 4.2 Hz, H6a′), 4.68 (3H, dd, J = 12.0, 2.4 Hz, H6b′), 5.07 (3H, d, J = 1.7 Hz, H1′), 5.69 (3H, dd, J = 3.3, 1.7 Hz, H2′), 5.91 (3H, dd, J = 10.1, 3.3 Hz, H3′), 6.11 (3H, t, J = 10.1 Hz, H4′), 7.23-7.41 (20H, m, Har meta), 7.41-7.58 (20H, m, Har para), 7.82-8.08 (20H, m, Har ortho).

NMR ¹³C (CDC13, 100 MHz) δ (ppm):

29.69 (C8), 29.95 (C5), 44.81 (C3a), 62.84 (C6′), 65.56 (C9), 66.05 (C2), 66.94 (C4′), 67.37 (C7), 68.11 (C4), 68.67 (C6), 68.78 (C5′), 70.14 (C3′), 70.53 (C2′), 71.46 (C3), 97.61 (C1′), 128.28 (CHar meta), 128.42 (CHar meta), 128.56 (CHar meta), 128.99 (Cqar), 129.09 (Cqar), 129.35 (Cqar), 129.72 (CHar ortho), 129.78 (CHar ortho), 129.83 (CHar ortho), 129.87 (Cqar), 133.03 (CHar para), 133.14 (CHar para), 133.41 (CHar para), 165.39 (COPh), 165.44 (COPh), 165.49 (COPh), 166.13 (COPh).

To a solution of alcohol (614 mg, 0.275 mmol) dissolved in 10 mL of AcOEt, are added 56 µL of an aqueous solution of KBr 0.5 M (0.028 mmol), the TEMPO (15 mg, 0.096 mmol), then at 0° C., 1.2 mL of NaOCl (0.84 mmol). After 3 hours at ambient temperature, the reaction is stopped and acidified with an aqueous solution of HCl at 5% (up to pH = 3), then 560 µL of NaO₂Cl (1.68 mmol) are added. The reaction medium is stirred for one night at ambient temperature (yellow colouration) then it is extracted three times with the AcOEt. The organic phase is washed with a saturated solution of NaCl, dried on MgSO4, filtered then vacuum concentrated in order to give 614 mg of carboxylic acid in the form of a white powder with a yield of 99%.

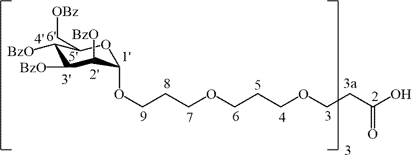

NMR ¹H (CDCl3, 400 MHz) δ (ppm):

1.83 (6H, p, J =, H5), 1.96 (6H, p, J = 6.2 Hz, H8), 3.51 (6H, t, J = 6.3 Hz, H6), 3.53 (6H, t, J = 6.1 Hz, H4), 3.56 (6H, m, H7), 3.64 (6H, s, H3), 3.66 (3H, td, J = 9.7, 6.4 Hz, H9b), 3.94 (3H, td, J = 9.7, 6.4 Hz, H9a), 4.47-4.41 (3H, m, H5′), 4.49 (3H. dd. J = 12.0. 4.2 Hz, H6b), 4.71 (3H, dd, J = 12.0, 2.4 Hz. H6a), 5.10 (3H, d, J = 1.7 Hz, H1′), 5.70 (3H, dd, J = 3.3, 1.7 Hz, H2′), 5.92 (3H, dd, J = 10.1, 3.3 Hz, H3′), 6.13 (3H, t, J = 10.0 Hz, H4′), 7.59-7.24 (36 H, m, Har), 8.11-7.82 (24 H, m, Har).

NMR ¹³C (CDCl3, 100 MH_{z}) δ (ppm):

29.60 (C8), 29.77 (C5), 52.86 (C3a), 60.34 (C2) 62.79 (C6′), 65.46 (C9), 66.85 (C4′), 67.31 (C7), 67.87 (C6), 68.59 (C4), 68.73 (C5′), 69.07 (C3), 70.13 (C3′), 70.49 (C2′), 97.55 (C1′), 128.24 (CHar), 128.38 (CHar), 128.52 (CHar), 128.92 (Cqar), 129.00 (Cqar), 129.27 (Cqar), 129.68 (CHar), 129.73 (CHar), 129.78 (CHar), 133.02 (CHar), 133.12 (CHar), 133.37 (CHar), 133.39 (CHar), 165.39 (COPh), 165.42 (COPh), 165.50 (COPh), 166.15 (COPh), 173.45 (COOH).

### Synthesis of the Tri-Mannosylated Lipid From Carboxylic Acid Diether and Carboxylic Acid Tri-Mannosylated Ligand (Oligo(Propylene Glycol)N Spacer Where N = 2 and a Polyethylene Glycol Spacer (PEG)M Where M = 5)

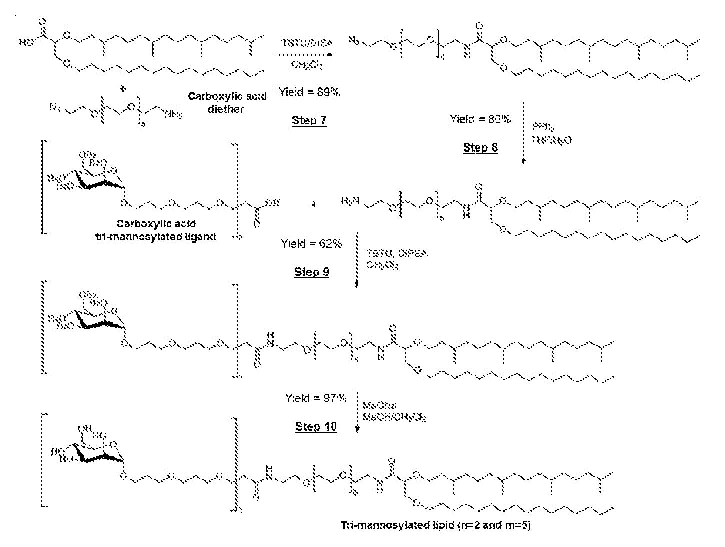

### Step 7: Introduction of the PEG Chain on the Carboxylic Acid Diether Lipid

To a mixture of the diether lipidcarboxylic acid (291 mg, 0.831 mmol) and TBTU (347 mg, 1.08 mmol) in anhydrous CH₂Cl₂ (15 mL), is added under an argon atmosphere the DIEA (188 µL, 1.08 mmol). The mixture is stirred at ambient temperature for 20 minutes under a nitrogen atmosphere. A solution of N3-PEG350-NH₂ (291 mg, 0.831 mmol) in anhydrous CH₂Cl₂ (5 mL) is added under a nitrogen atmosphere. The reaction medium is stirred at ambient temperature for 12 hours under a nitrogen atmosphere. An aqueous solution of hydrochloric acid 1N is added and the organic phase (pH = 1) is washed with water. The organic phases are grouped together, dried (MgSO₄), filtered and concentrated under reduced pressure. The residue is purified by chromatography on silica gel (eluent CH₂Cl₂/MeOH: 98/2) in order to isolate the azide coupling product (700 mg, 89%) in the form of a colourless oil.

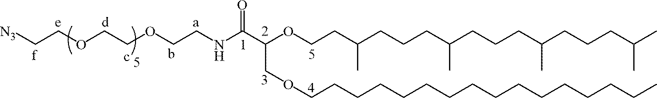

NMR ¹H (CDCl3, 400 MHz) δ (ppm):

0.83-0.87 (m, 18H, 6 CH₃), 1.04-1.78 (m, 52H, 24 CH₂, 4 CH), 3.37-3.40 (m, 2H, H-f), 3.41-3.50 (m, 4H, H-a, H-4), 3.53-3.57 (m, 4H, H-b, H-5), 3.58-3.69 (m, 23H, PEG, H-e, H-3α), 3.75-3.78 (m, 1H, H-3β), 3.88-3.90 (dd, J = 2.51, 5.92 Hz, 1H, H-2), 7.02-7.05 (m, 1H, NHCO).

NMR ¹³C (CDCl3, 100 MHz) δ (ppm):

14.10 (CH₃), 19.58, 19.65, 19.72 (3 CH₃), 22.60, 22.69 (2 CH₃), 22.66, 24.36, 24.47, 24.78, 26.03 (5 CH₂), 27.94, 29.84, 32.76, 32.78 (4 CH), 29.34, 29.46, 29.52, 29.63, 29.68, 31.89, 37.26, 37.36, 37.39, 37.45, 37.50, 39.32 (19 CH₂), 38.67 (C-a), 50.63 (C-f), 69.72 (C-5), 69.83 (C-b), 70.3-70.7 (C-PEG, C-e), 71.47 (C-3), 71.68 (C-4), 80.48 (C-2), 170.57 (C-1).

### Step 8: Reduction of the Azide Function

To a solution of azide (212 mg, 0.224 mmol) in a THF/H₂O mixture (25 mL, 1/1) is added the triphenylphosphine (88 mg, 0.337 mmol) by portion. The mixture is stirred at ambient temperature for 18 h. After concentration under reduced pressure, the residue is purified on silica gel chromatography (CH₂Cl₂/MeOH: 9/1) in order to isolate the amine (164 mg, 80%) in the form of a light yellow oil.

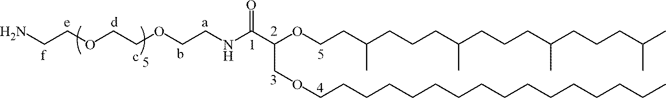

NMR ¹H (CDCl3, 400 MHz) δ (ppm):

0.82-0.88 (m, 18H, 6 CH₃), 0.99-1.69 (m, 52H, 24 CH₂, 4 CH), 3.16-3.18 (m, 2H, H-f), 3.39-3.49 (m, 4H, H-a, H-4), 3.54-3.57 (m, 4H, H-b, H-5), 3.59-3.70 (m, 18H, PEG), 3.72-3.75 (m, 3H, H-c, H-3α), 3.76-3.77 (m, 1H, H-3β), 3.87-3.90 (m, 3H, H-2, H-e), 7.04-7.06 (m, 1H, NHCO).

NMR ¹³C (CDCl3, 100 MHz) δ (ppm):

14.10 (CH₃), 19.58, 19.65, 19.72 (3 CH₃), 22.61, 22.71 (2 CH₃), 22.66, 24.34, 24.45, 24.76, 26.00 (5 CH₂), 27.95, 29.77, 29.85, 32.79 (4 CH), 29.32, 29.45, 29.54, 20.61, 29.66, 31.88, 37.23, 37.34, 37.37, 37.43, 39.31 (19 CH₂), 38.70 (C-a), 40.45 (C-f), 66.89 (C-e), 69.72-70.53 (C-PEG, C-5, C-b), 71.45 (C-3), 71.70 (C-4), 80.47 (C-2), 170.63 (C-1).

### Step 9: Coupling Between the Carboxylic Acid Tri-mannosylated Ligand and the Amine lipid

To a mixture of the carboxylic acid tri-mannosylated ligand (575 mg, 0.257 mmol) and of TBTU (124 mg, 0.386 mmol) in anhydrous CH₂Cl₂ (70 mL), is added under an argon atmosphere the DIEA (112 µL, 0.643 mmol). The mixture is stirred at ambient temperature for 20 minutes under a nitrogen atmosphere. A solution of the amide lipid (335 mg, 0.365 mmol) in anhydrous CH₂Cl₂ (50 mL) is added under a nitrogen atmosphere. The reaction medium is stirred at ambient temperature for 12 hours under a nitrogen atmosphere. An aqueous solution of hydrochloric acid 1N is added (pH = 1) and the organic phase is washed with water. The organic phases are grouped together, dried (MgSO₄), filtered and concentrated under reduced pressure. The residue is purified by chromatography on silica gel (eluent Cyclohexane/AcOEt: 1:1 + 2% MeOH) in order to isolate the azide coupling product (500 mg, 62%) in the form of a translucent solid.

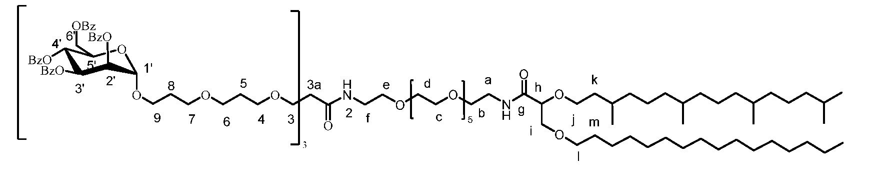

NMR ¹H (CDCl3, 400 MHz) δ (ppm):

0.83-0.89 (m, 21H, CH₃), 1-1.59 (m, 68H, alkyl chains), 1.79-1.86 (6H, q, H5), 1.93-1.99 (6H, q, H8), 3.40-3.68 (65H, m, H3, H4, H6, H7, H9b, Hia, Hj, Hl, HPEG), 3.75-3.78 (m, 1H, Ch)3.88-3.95(4H, m, H9a, Hib), 4.40-4.44 (3H, m, H5′), 4.48 (3H, dd, J = 4.1, 12.0 Hz, H6b′), 4.69 (3H, dd, J = 2.1, 12.1 Hz, H6a′), 5.09 (3H, d, J = 1.8 Hz, H1′), 5.69 (3H, dd, J = 1.6, 3.4 Hz, H2′), 5.91 (3H, dd, J = 3.2, 10.3 Hz, H3′), 6.11 (3H, t, J = 10.3 Hz, H4′), 7.24-8.11 (60H, m, Har).

NMR ¹³C (CDCl3, 100 MHz) δ (ppm):

14.12, 19.62, 19.68, 19.75, 22.63, 22.69 (CH₃), 22.72-29.56 (CH₂), 29.65 (C8), 29.70 (C5), 29.74, 29.84( C5), 29.91, 31.10, 31.43, 31.63, 31.92, 32.80, 53.03 (C3a), 60.40 (C2), 62.85 (C6′), 65.53 (C9), 66.91 (C4′), 67.36 (C7), 67.92 (C6), 68.61 (C4), 68.78 (C5′), 68.95 (C3), 70.15 (C3′), 70.55 (C2′), 80.53 (Ch), 97.64 (C1′), 128.30 (CHar), 128.44 (CHar), 128.57 (CHar), 129.00 (Cqar), 129.10 (Cqar), 129.35 (Cqar), 129.73 (CHar), 129.79 (CHar), 129.84 (CHar), 129.88 (CHar), 133.05 (CHar), 133.16 (CHar), 133.43 (COPh), 165.4 (COPh), 165.50 (COPh), 166.14 (COPh), 170.69 (CONH).

### Step 10: Synthesis of the Tri-mannosylated Lipid by Deprotection of the Hydroxyls of The mannose moieties

To a solution of the tri-mannosylated lipid in benzoylated form (435 mg, 0.139 mmol) in a CH₂Cl₂/MeOH mixture (100 mL, 1/1) is added a solution of MeONa in the methanol (5.3 M, 48.8 µL, 0.258 mmol) freshly prepared. The reaction medium is stirred for one night at ambient temperature. The mixture is neutralised thanks to the adding of Amberlite IR-120 H+ resin and the resin is filtered on cotton. After evaporation of the solvents under reduced pressure, a viscous light grey yellowish product is isolated (255 mg, 97%) corresponding to the target tri-mannosylated lipid. 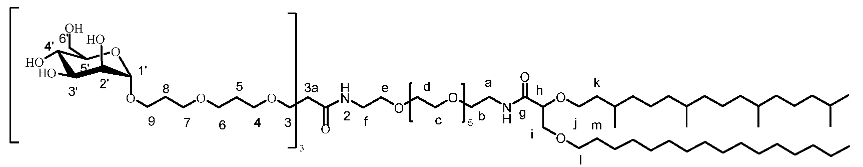 NMR ¹H (MeOD+CDCl3, 70/30, 400 MHz) δ (ppm):

0.84-0.89 (m, 21H, CH3), 1.04-1.56 (m, 68H, alkyl chains), 1.79-1.84 (12H, m, H5, H8), 3.31-3.88 (88 H, m, H3, H4, H6, H7, H9, Hi, Hj, Hl, Ch, H3′, H4′, H5′, H6′, HPEG), 4.74 (3H, s, H1′), 7.55 (NH, s).

NMR ¹³C (MeOD+CDCl3, 70/30, 100 MHz) δ (ppm):

13.66, 19.26, 19.30, 19.33, 19.37, 19.40, 19.44, 22.22, 22.31, 22.50, 24.24, 24.31, 24.65, 25.92, 27.83, 29.21, 29.32, 29.39; 29.48, 29.50, 29.53, 29.64, 29.68, 29.72, 31.27, 31.78, 32.62, 32.64, 32.67, 36.59, 36.65, 36.72, 37.13, 37.23, 37.25, 37.30, 37.36, 37.39, 38.67, 38.95, 39.25, 52.25, 61.45, 64.22, 67.06, 67.52, 67.69, 68.41, 69.43, 69.48, 69.53, 69.61, 70.09, 70.11, 70.23, 70.33, 70.35, 70.37, 70.70, 71.11, 71.15, 71.33, 71.65, 72.56, 80.34, 80.29, 100.08 (C1′), 171.49 (CONH), 173.69 (CONH).

HRMS_{C194H1182N2O34}: [M+Na]+: m/z theoretical = 1906.24662, measured = 1906.2448

### Example 2: Preparation of Liposomes and of Lipopolyplexes (LPR) According to the Invention Comprising Tri-Mannosylated Lipids (LPR-triMN)

### Lipids Used for the Preparation of the Various Liposomes

lipid KLN25 (O,O-dioleyl-N-[3N-(Nmethylimidazoliumbromide)propylene] phosphoramidate) of formula:

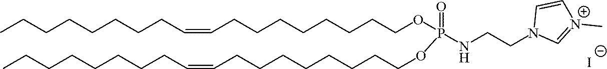

described by Mével et al. (Mével et al., (2008) Synthesis and Transfection Activity of New Cationic Phosphoramidate Lipids: High efficiency of an Imidazolium derivative. ChemBioChem. 9, 1462-1471);
lipid MM27 (O,O-dioleyl(-N-(histamine)phosphoramidate) of formula:

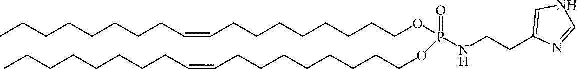

described by Mevel et al., (Mével et al., (2008) Novel neutral imidazole-lipophosphoramides for transfection assays. Chem. Comm. 21:3124-3126);
1-O-carboxyl-2-O-Phytanyl-3-O-hexadecane-sn-glycerol (diether lipid having a carboxylic acid function) of formula:

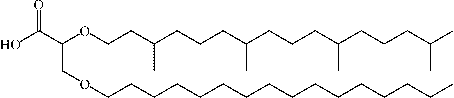

mono-mannosylated lipid (β-D-mannopyranosyl-N-dodecylhexadecanamide) of formula:

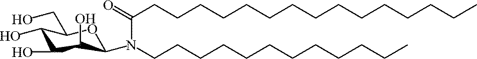

described by Perche et al., (Perche et al., (2011) Selective gene delivery in dendritic cells with Mannosylated and Histidylated Lipopolyplexes. J Drug Targeting. 19: 315-325);
lipid marked with fluorescein (Lip-Flu) of formula:

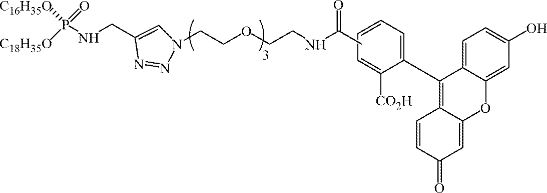

described by Berchel et al., (Berchel et al., (2011) Modular Construction of Fluorescent Lipophosphoramidates by Click Chemistry. Eur. J. Org. Chem 31: 6294-6303);
lipid marked with rhodamine (Lip-Rho) of formula:

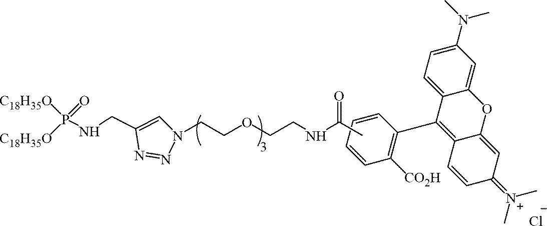

described by Berchel et al., (Berchel et al., (2011) Modular Construction of Fluorescent Lipophosphoramidates by Click Chemistry. Eur. J. Org. Chem 31: 6294-6303).

### Liposomes

The liposomes are prepared according to the method of hydrating a dry lipid film and dialysed against a buffer HEPES 10 mM, pH 7.4 (Pichon C, Midoux P. (2013) Mannosylated and Histidylated LPR Technology for Vaccination with Tumor Antigen mRNA. Methods Mol Biol. 969:247-74).

The compositions of liposomes used are:
bare liposomes: liposomes constituted of a mixture of KLN25 and MM27 lipids at 50%-50% in mole percent with respect to the total number of moles of lipids;
diether liposomes or bare diether liposomes: liposomes constituted of a mixture of KLN25, MM27 lipids and of 1-O-carboxyl-2-O-Phytanyl-3-O-hexadecane-sn-glycerol (diether lipid) at 47.5% - 47.5% - 5% respectively;
mono-mannosylated liposomes (MN): liposomes constituted of a mixture of KLN25, MM27 lipids and mono-mannosylated lipid (β-D-mannopyranosyl-N-dodecylhexadecanamide) at 47.5% - 47.5% - 5% respectively;
mono-mannosylated diether liposomes: liposomes constituted of a mixture of KLN25, MM27 lipids, mono-mannosylated lipid and 1-O-carboxyl-2-O-Phytanyl-3-O-hexadecane-sn-glycerol (diether lipid) at 45% - 45% - 5% - 5% respectively;
tri-mannosylated liposomes (triMN): liposomes constituted of a mixture of KLN25, MM27 lipids and tri-mannosylated lipids of the invention (of which the synthesis is described in the example 1) at 47.5% - 47.5% - 5% respectively.

The compositions of the fluorescent liposomes used are:
bare-Flu liposomes: liposomes constituted of a mixture of KLN25, MM27 lipids and Lip-Flu at 49.75% - 49.75% - 0.5% respectively;
bare-Rho liposomes: liposomes constituted of a mixture of KLN25, MM27 lipids and Lip-Rho at 49.75% - 49.75% - 0.5% respectively;
diether-Flu liposomes: liposomes constituted of a mixture of KLN25, MM27 lipids, of 1-O-carboxyl-2-O-Phytanyl-3-O-hexadecane-sn-glycerol and Lip-Flu at 47.25% - 47.25% - 5 % - 0.5% respectively;
diether-Rho liposomes: liposomes constituted of a mixture of KLN25, MM27 lipids, of 1-O-carboxyl-2-O-Phytanyl-3-O-hexadecane-sn-glycerol and Lip-Rho at 47.25% - 47.25% - 5 % - 0.5% respectively;
Flu mono-mannosylated liposomes: liposomes constituted of a mixture of KLN25, MM27 lipids, mono-mannosylated lipid (β-D-mannopyranosyl-N-dodecylhexadecanamide) and Lip-Flu at 47.25% - 47.25% - 5% - 0.5% respectively;
Rho mono-mannosylated liposomes: liposomes constituted of a mixture of KLN25, MM27 lipids, mono-mannosylated lipid and Lip-Rho at 47.25% -47.25% - 5% - 0.5% respectively;
dietherFlu mono-mannosylated liposomes: liposomes constituted of a mixture of KLN25, MM27 lipids, mono-mannosylated lipid, of 1-O-carboxyl-2-O-Phytanyl-3-O-hexadecane-sn-glycerol and Lip-Flu at 44.75% - 44.75% - 5% -5% - 0.5% respectively;
diether-Rho mono-mannosylated liposomes: liposomes constituted of a mixture of KLN25, MM27 lipids, mono-mannosylated lipid, of 1-O-carboxyl-2-O-Phytanyl-3-O-hexadecane-sn-glycerol and Lip-Rho at 44.75% - 44.75% - 5% -5% - 0.5% respectively;
Flu tri-mannosylated liposomes: liposomes constituted of a mixture of KLN25, MM27 lipids, tri-mannosylated lipids of the invention (of which the synthesis is described in the example 1) and Lip-Flu at 47.25% - 47.25% - 5% - 0.5% respectively;
Rho tri-mannosylated liposomes: liposomes constituted of a mixture of KLN25, MM27 lipids, tri-mannosylated lipids of the invention and Lip-Rho at 47.25% -47.25% - 5% - 0.5% respectively.

### Cationic Polymer

The mRNA was complexed with the partially histidinylated polylysine comprising one molecule of PEG 5 kDa (PEG-HpK) of which the synthesis is described in (Pichon C, Midoux P. (2013) Mannosylated and Histidylated LPR Technology for Vaccination with Tumor Antigen mRNA. Methods Mol Biol. 969:247-74).

### Lipopolyplexes (LPR)

The various LPR are prepared according to the method described in (Pichon C, Midoux P. (2013) Mannosylated and Histidylated LPR Technology for Vaccination with Tumor Antigen mRNA. Methods Mol Biol. 969:247-74).

In particular, for an in vitro transfection, the LPR are obtained in the following way. Briefly, 15 µg of PEG-HpK (in 10 µL of buffer 10 mM HEPES pH 7.4) are first added to the mRNA (5 µg in 25 µL of buffer 10 mM HEPES pH 7.4). The whole is vortexed for 4 sec then allowed to stand for 30 min at 20° C. The LPR are then formed by adding 10 µg of liposomes (5 µL to 5.4 mM in 10 mM HEPES buffer, pH 7.4) by stirring the solution via back-and-forth pipetting. The solution is allowed to stand for 15 minutes at 20° C. before use. For the in vitro transfection, the solution volume is adjusted to 1 ml with the medium without serum.

For an in vivo injection, the LPR are obtained by adding beforehand 50 µl (50 µg) of mRNA in a 1.5 ml Eppendorf tube containing 160 µl of sterile buffer HEPES 10 mM at pH 7.4 prepared in water without endonuclease. 100 µl (150 µg) of the solution of PEG-HpK are then added and mixed 4 seconds. After 30 min at 20° C., 50 µL of liposomes (100 µg in sterile buffer HEPES 10 mM at pH 7.4 prepared in water without endonuclease) are added to the polymer/mRNA complex and gently mixed by pipetting. The solution is allowed to stand for 15 min at 20° C. For the in vivo injection, the solution is adjusted to a final concentration in saccharose of 5% by adding 40 µl of a solution of saccharose at 50% prepared in water without endonuclease. The mice are injected with 100 µl of solution.

Unless indicated otherwise, the lipopolyplexes (called LPR) used in the experiments described contain RNA control single strand PolyU (ssPolyU).

The various LPR (bare LPR, diether LPR or bare diether LPR, LPR-MN, LPR-MN diether, LPR-triMN) are obtained by mixing the corresponding liposomes (bare liposomes, diether liposomes or bare diether liposomes, liposomes MN, liposomes MN diether, liposomes triMN) with a cationic polymer (RNA complexed with the partially histidinylated polylysine and comprising one molecule of PEG 5 kDa).

### Example 3: Biological Use of the Liposomes of the Invention

### Material and Methods

### Cell Lines

The cell line 293T is a cell line derived from transformed human embryonic kidney cells.

The cell line 293T DC-SIGN corresponds to genetically modified cells 293T to express DC-SIGN, a type C lectin.

MoDCs cells are human monocyte-derived dendritic cells.

PBMCs cells are mononuclear cells of the blood.

PanDCs cells are human dendritic cells identified within the other cells of the peripheral blood (PBMCs).

### ELISpot Interferon-y

The secretion of interferon-y by the lymphocytes T coming from the spleen (spleen cells) is analysed using the kit IFN-γ ELISpot^{PLUS} kit (Mabtech, Sweden). Briefly the spleen cells to be analysed are incubated in an ELISpot plate in the presence of the antigen E7₄₉₋₅₇ or of concanavalin A (positive control) or of culture medium (negative control). The interferon-y secreted is captured by antibodies attached to the plate. After an incubation of 36 h at 37° C., the plate is washed and the cells are removed. The plate is then incubated with a biotinylated antibody directed against interferon-y then with the streptavidin coupled to the alkaline phosphatase. The presence of spots corresponding to a secretion of interferon-y is detected after incubation with the reaction buffer BCIP/NBT. The spots are analysed by an ELISpot plate reader.

### qRT-PCR

The total RNA were extracted from samples of skin using the ReliPrep RNA Tissue Miniprep system kit (Z6112, Promega), by following the supplier’s instructions. The RNA were then retrotranscribed in cDNA using the GoScript Reverse Transcription system kit (A5000, Promega), by following the supplier’s instructions. The quantitative PCR reactions in real time SYBR green (qRT-PCR) were carried out on the iGenSeq platform (Hôpital de la Pitié-Salpêtrière, Paris, France) with the primers described in Table 1. The results were analysed with the 1536 Lightcycler software (Roche, Basel, Switzerland) and the expression of the genes was quantified by the relative method of ΔCT, standardised by the expression of the reference genes β-actin and GAPH.

TABLE 1

Primers used for the reactions of RT-qPCR

| Gene | Primer | Sequence | SEQ ID NO |
|---|---|---|---|
| CCR7 | Sense | ACTCTCCATCCACCGAATTG | 1 |
| | Antisense | CCTCATGTCAACCTGACTGG | 2 |
| CXCR4 | Sense | TCCAGAATGTGTGGTAAATTGAA | 3 |
| | Antisense | TCGGAATGAAGAGATTATGCAG | 4 |
| IL1β | Sense | AGTTGACGGACCCCAAAAG | 5 |
| | Antisense | AGCTGGATGCTCTCATCAGG | 6 |
| MMP9 | Sense | CCAGAGGTAACCCACGTCAG | 7 |
| | Antisense | CTTCAAGTCGAATCTCCAGACA | 8 |
| PGE2R1 | Sense | TGGCTTCATATTCAAGAAACCAG | 9 |
| | Antisense | GGTACACGCGTGACTTTCG | 10 |
| β-actin | Sense | AAGTCCCTCACCCTCCCAAAAG | 11 |
| | Antisense | AAGCAATGCTGTCACCTTCCC | 12 |
| GAPDH | Sense | GTATTGGGCGCCTGGTCACC | 13 |
| | Antisense | CGCTCCTGGAAGATGGTGATGG | 14 |

### The LPR-triMN Target Cells That Express Receptors of the Lectin Type

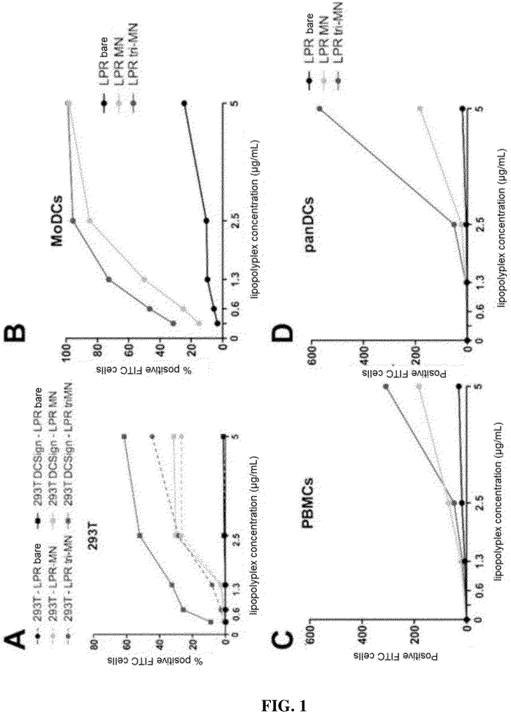

In a first experiment, different cell lines were incubated with different concentrations of bare lipopolyplexes (LPR), of mono-mannosylated lipopolyplexes (LPR-MN) or of tri-mannosylated lipopolyplexes (LPR-triMN). The lipopolyplexes all comprise a fluorescent lipid coupled to the fluorescein. The cell lines tested express type C lectins on their surface, able to bind the sugar residues mannose, galactose or fucose. The binding of the lipopolyplexes on these receptors is analysed by flow cytometry. The tri-mannosylated lipopolyplexes target the cells 293T DCSign (FIG. 1A), the cells MoDCs (FIG. 1B), the cells PBMCs (FIG. 1C) and the cells panDCs among the PBMCs (FIG. 1D). To a lesser degree, the mono-mannosylated lipopolyplexes (LPR-MN) are also capable to bind these cells (FIG. 1). The bare lipopolyplexes do not comprise lipids having a mannose group and do not target any of these cells regardless of the concentration of lipopolyplexes tested (FIG. 1).

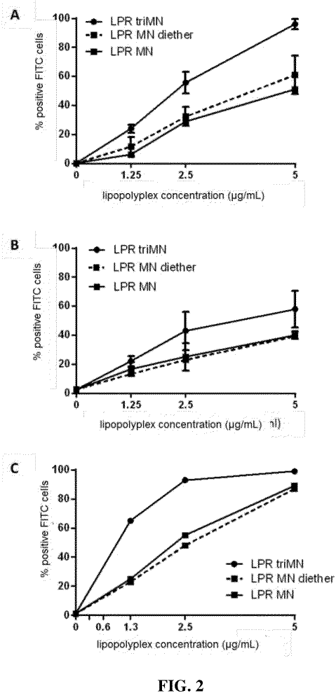

In a second experiment, three different cell lines were incubated with increasing concentrations of mono-mannosylated lipopolyplexes (LPR-MN), diether mono-mannosylated lipopolyplexes (LPR-MN diether) and tri-mannosylated lipopolyplexes (LPR-triMN). FIG. 2 shows that the LPR-MN and LPR-MN diether target in an identical way the dendritic cells of a murine line, the murine spleen cells and the cells MoDCs. The LPR-triMN are bound to these three types of cellules significantly more substantially, even at a low concentration (1.25 µg/ml).

The presence of tri-mannosylated lipids of the invention therefore provides the LPR-triMN lipopolyplexes the capacity to bind to the cells expressing lectin type receptors. This property is specific to the tri-mannosylated lipopolyplexes of the invention, and lipopolyplexes comprising mono-mannosylated lipids (LPR-MN) or mono-mannosylated lipids and diether lipids (diether LPR-MN) have a reduced binding capacity.

### The LPR-triMN Activate the Dendritic Cells

The dendritic cells MoDCs are placed in a culture in the presence of LPS or of increasing doses of LPR-MN or LPR-triMN comprising lipids marked with fluorescein for 6h. The analysis via cytometry makes it possible to identify the fluorescent cells MoDCs that have captured the LPR. Moreover the expression of the activation markers HLA-DR, CD80 and CD83 is also measured.

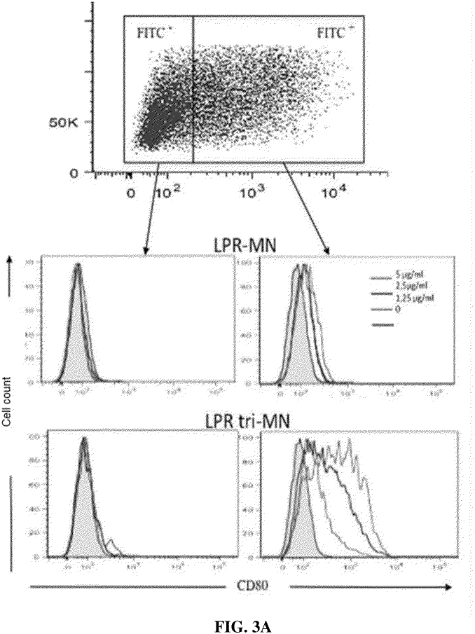

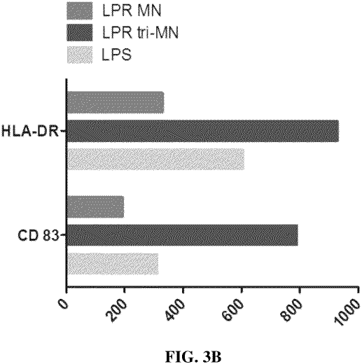

The fluorescent cells MoDCs that have captured the LPR-MN do not express activation markers significantly with respect to the activation control LPS (FIGS. 3A and 3B). The fluorescent cells MoDCs that have captured the LPR-triMN express the activation markers HLA-DR, CD80 and CD83 in a dose-dependent manner (FIG. 3A) and significantly with respect to the activation control LPS (FIG. 3B).

The presence of tri-mannosylated lipids of the invention therefore provides the LPR-triMN with the intrinsic property of activating the dendritic cells to which they are bound. The activated dendritic cells expressing co-stimulation molecules such as CD80 allow in turn for the activation of the lymphocytes T CD4+ and CD8+. The LPR-triMN are therefore capable of stimulating an immune response even though they do not contain any immunogenic molecule of interest.

### The LPR-triMN Have an Adjuvant Effect

In a first step, the dendritic cells MoDCs ate placed in culture in the presence of LPR-MN containing mRNA GFP for 6 h. After these 6 h, the cells MoDCs are put into contact for an additional 12 h with either LPS (positive control of activation of MoDCs), or with LPR-MN or LPR-triMN. The lipopolyplexes used for this second stimulation do not contain encoding RNA but contain RNA single strand PolyU (ssPolyU). The expression of the GFP and of the activation markers HLA-DR, CD80 and CD83 is measured by cytometry after 18 h of culture in total.

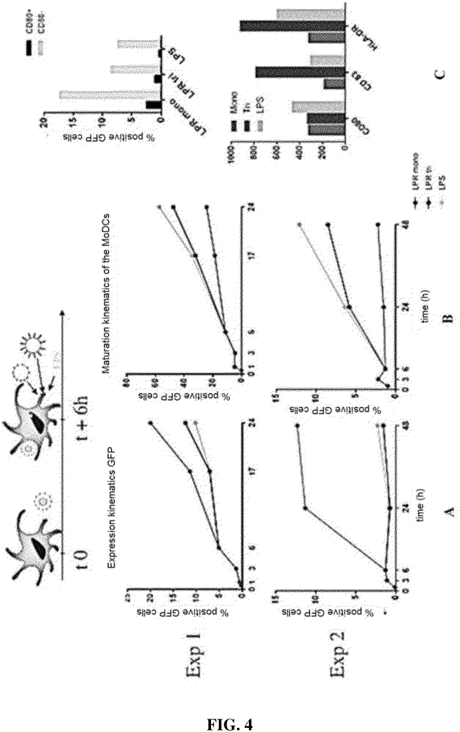

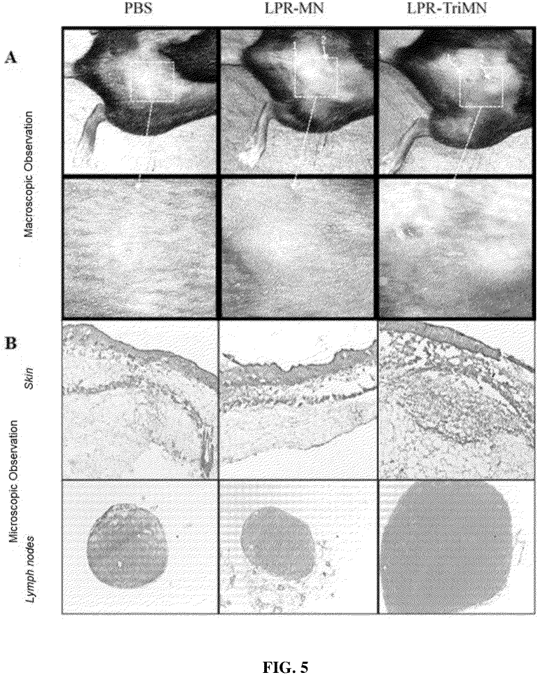

FIG. 4 shows that the first incubation of the cells MoDCs with LPR-MN induces the expression of GFP. The secondary incubation of the same cells with LPS or with LPR-triMN ssPolyU causes the level of expression of the GFP (FIG. 4A) to drop and induces the expression of the activation markers CD80 (FIG. 4B), CD83 and HLA-DR (FIG. 4C). The adding of LPR-MN does not induce the activation of the dendritic cells, the latter continue to express the GFP in a prolonged manner.

The incubation of dendritic cells with LPR-triMN ssPolyU makes it possible to induce the activation of these cells. This experiment confirms that the LPR-triMN have an adjuvant effect provided by the presence of tri-mannosylated lipids.

### The LPR-triMN Have an in Vivo Adjuvant Effect

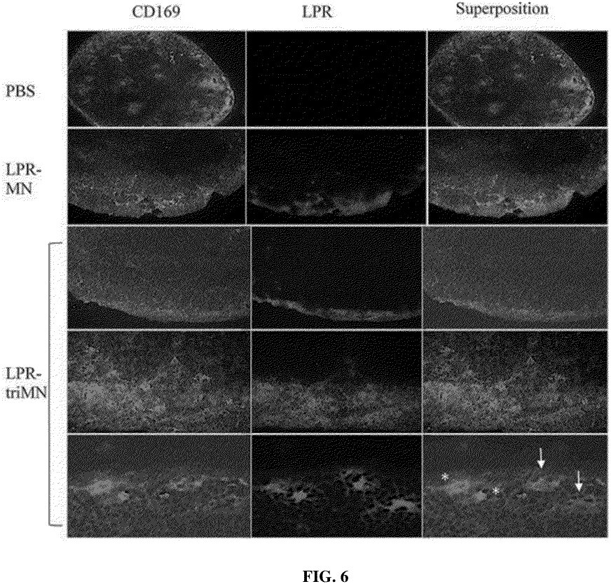

In a first experiment, mice were intradermally injected with PBS, LPR-MN or LPR-triMN. FIG. 5A shows the injection of LPR-triMN induces a local inflammatory reaction on the site of the injection. The microscopic analysis of the site of injection (FIG. 5B) shows that the of LPR-triMN induces an augmentation in the volume of the inguinal draining lymph nodes.

In addition, analyses via qRT-PCR conduced on skin samples show a significant increase in the number of transcription products of genes CCR7 and CXCR4 (results not shown). These receptors are known to play a role in the migration of dendritic cells of the skin to the lymph nodes (Förster et al., (1999) CCR7 coordinates the primary immune response by establishing functional microenvironments in secondary lymphoid organs. Cell 99:23-33; Kabashima et al., (2007) CXCL12-CXCR4 Engagement Is Required for Migration of Cutaneous Dendritic Cells. Am J Pathol. 171:1249-57). The results of qRT-PCR also show an increase in the transcription of genes MMP9, IL1β and PGE2R1 (results not presented), that are involved in the inflammatory response.

In a second experiment, mice were intradermally injected on the foot arch with PBS, LPR-MN or LPR-triMN comprising a lipid marked with rhodamine. The popliteal draining lymph nodes of the mice were taken 6h after injection then marked with an antibody anti-CD169 (siglec-1). The analysis via fluorescence microscopy thus makes it possible to view the location of the LPR (rhodamine signal) and of the macrophages of the subcapsular sinus (signal CD169+). FIG. 6 shows that the bare LPR, LPR-MN and LPR-triMN, can be detected in the popliteal lymph nodes, on the cortical zone where dendritic cells (not marked) are also located. A portion of the LPR-triMN can also be detected in the unmarked zones by CD169 which indicated that other cells can capture the LPR-triMN. Note that dendritic cells are found in all of the lymph node zones.

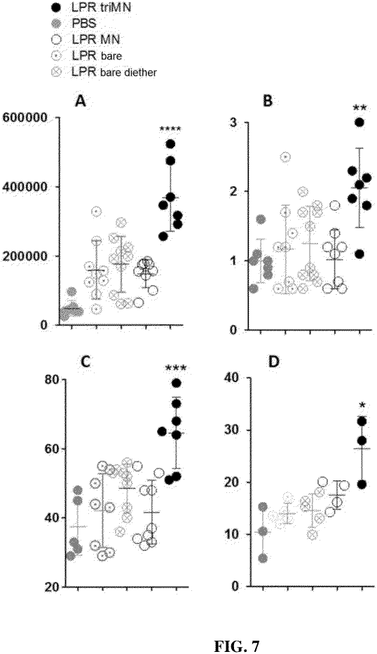

Finally in a third experiment, mice were intradermally injected on the foot arch with PBS, bare LPR, bare diether LPR, LPR-MN or LPR-triMN. The popliteal lymph nodes of the mice were taken 24 h after injection. The presence in the popliteal draining lymph nodes of dendritic cells (DCs), and more particularly of activated dendritic cells expressing the marker Ly6C (Ly6C+ cells) or Ly6G (Ly6G+ cells), was analysed by flow cytometry. FIG. 7 shows that the injection of LPR-triMN induces not only a significant increase in the number and in the percentage of dendritic cells (FIGS. 7A and 7B) but also in the percentage of activated (FIG. 7C) and inflammatory (FIG. 7D) dendritic cells among the dendritic cells contained in the draining lymph nodes of the injected mice. The number of dendritic cells, activated or not, is not significantly modified after the injection of bare LPR, of bare diether LPR or of LPR-MN with respect to the control (PBS).

All of these experiments show that the LPR-triMN comprising the tri-mannosylated lipid of the invention can induce an immune response (recruiting and activation of the dendritic cells in the lymph nodes draining the injection site) when they are injected into mice. In these experiments, the LPR-triMN contain RNA single strand PolyU (ssPolyU) and are not used as a vector for the introduction of immunogenic molecules of interest. These experiments show that the lipopolyplexes triMN have an intrinsic in vivo adjuvant effect.

### The LPR-triMN Induce a Better Specific T-lymphocyte Response in Vitro in Humans

Dendritic cells MoDCs coming from HLA-A2 donors were incubated for 24 h with bare LPR, LPR-MN or LPR-triMN containing mRNA of oncoprotein E7 of the virus HPV16 (mRNA E7) or the RNA non-encoding single strand PolyU (ssPolyU). The cells MoDCs where then used to sensitise T CD3+ autologous lymphocytes in co-culture for 3 days. After 3 days of co-culture the T lymphocytes were placed in a medium enriched with IL-7 and IL-15. At D7, the sensitised T lymphocytes were put back in the presence of MoDCs that were loaded beforehand with peptides E7 restricted to HLA-A2 (allele HLA2) for 16 h. Finally the exocytosis was inhibited for 4 h before conducting an intracellular marking of interferon-γ (INF-γ) analysed by cytometry. A detection of the expression of membrane markers CD3, CD4 and CD8 was also conducted.

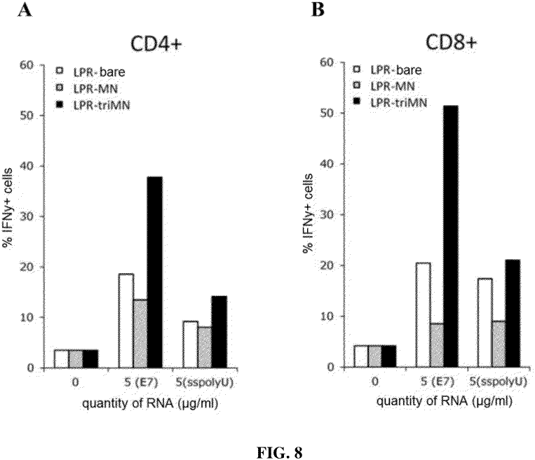

FIG. 8 shows that the LPR-triMN that do not contained encoding RNA induce a secretion of INF-γ in the lymphocytes CD4+ (FIG. 8A) and the lymphocytes CD8+ (FIG. 8B) slightly greater than that induced by the bare LPR. This observation confirms the intrinsic adjuvant effect of the LPR-triMN. The LPR-triMN containing mRNA E7 are capable of inducing a secretion of INF-γ in lymphocytes CD4+ (FIG. 8A) and lymphocytes CD8+ (FIG. 8B) that is significant greater than that induced by the bare LPR or LPR-MN. The LPR-MN induce only a low secretion of INF-γ, whether they contain RNA ssPolyU or mRNA E7.

This experiment therefore shows that the LPR-triMN, used as a vector for the vaccination “anti-E7”, are capable of inducing a specific “anti-T7” reaction.

### The LPR-triMN Induce a Better Immune Response in Vivo

Mice vaccination experiments were conducted with lipopolyplexes containing mRNA encoding oncoprotein E7 of the virus HPV16. These mice were vaccinated with an equimolar mixture of LPR-E7 and of LPR-E7-DC-LAMP which makes it possible to obtain a better response to the vaccination (Mockey et al. (2007) mRNA-based cancer vaccine: Prevention of B16 melanoma progression and metastasis by systemic injection of MART1 mRNA histidylated lipopolyplexes. Cancer Gene Therapy 14, 802-814). Indeed DC-LAMP is a glycoprotein of the membrane of the lysosomes and of the late endosomes, which play a role in the loading of peptides on CMH-II, making it possible to actively induce responses Th1. The use of a chimeric nucleic acid sequence (by fusion of the sequence encoding E7 and of the sequence encoding DC-LAMP) makes it possible to orient E7 to the path of the CMH-II and to improve the immune response.

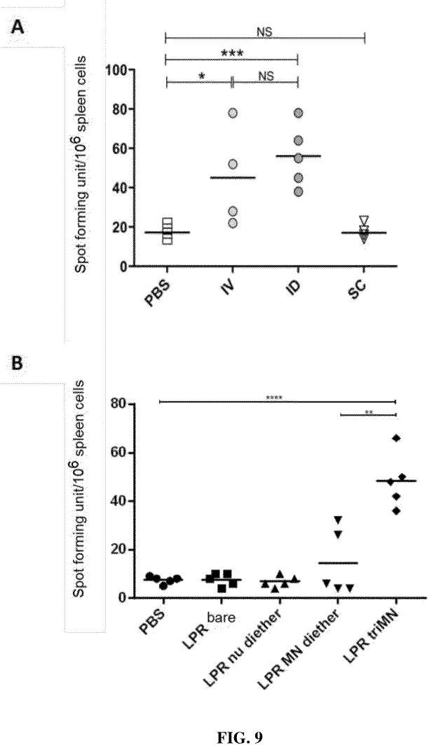

LPR containing RNA non-encoding single strand polyU (ssPolyU) were used as a control. The injections were conducted at D0, D7 and D15. At D21 the mice were sacrificed and their spleens were taken. The spleen cells isolated as such were incubated in the presence of peptides E7 and their secretion of INF-γ was analysed by ELISpot. Various modes of vaccination were compared. Groups of 5 mice were thus vaccinated with 21 µg of LPR-triMN-ssPolyU (control) intravenously or with LPR-triMN-E7/E7-DC-LAMP: 21 µg intravenously, 7 µg intradermally or 7 µg subcutaneously. FIG. 9A shows that the intravenous or intradermal vaccinations induce a significant antigen-specific immune response E7 in the vaccinated mice. The intradermal path seems however more effective and more advantageous as it induces an immune response of intensity that is equivalent to that induced intravenously, but after injection of 7 µg of LPR compared to 21 µg intravenously.

Similar experiments have been conducted to compare the effect induced by the injection of different LPR. Mice were vaccinated at D0 and D2 with PBS (control) or different LPR (bare LPR, bare diether LPR, diether LPR-MN, LPR-triMN) containing mRNA E7. At D14 the mice were sacrificed and their spleens were taken. The spleen cells isolated as such were incubated in the presence of peptides E7 and their secretion of INF-γ was analysed via ELISpot. FIG. 9B shows that the LPR-triMN induce a specific T response that is significantly greater than that induced by the diether LPR-MN. The injection of bare LPR or of LPR diether (bare diether LPR) does not induce a specific T response that is significant with respect to the control (PBS).

Furthermore, the mannosylated LPR, although positively charged, do not accumulate in the lungs (results not presented).

The LPR-triMN are therefore effective vaccination vectors for the induction of a specific T response to an antigen of interest in the vaccinated subjects.

### The LPR-triMN Have a Therapeutic Vaccine Effect in a Murine Model of HPV-induced cancer

The therapeutic effect of a vaccination with LPR-E7 was evaluated in groups of 5 mice to which were administered 50,000 cells of the syngeneic tumoural line TC-1 by intradermal injection in the ectopic position (left side). These tumour cells express oncoprotein E7.

The mice then received two intradermal injections (7 days and 9 days after the inoculation of the tumour cells) of PBS (negative control) or of 7 µg of: LPR-MN-ssPolyU, LPR-MN-E7/E7-DC-LAMP, LPR-triMN-ssPolyU or LPR-triMN-E7/E7-DC-LAMP.

The tumoural growth was evaluated every 2 days by measuring with a calliper, according to the formula (L×l²)/2. Animals for which the tumoural volume becomes critical (>2000 mm²) were sacrificed.

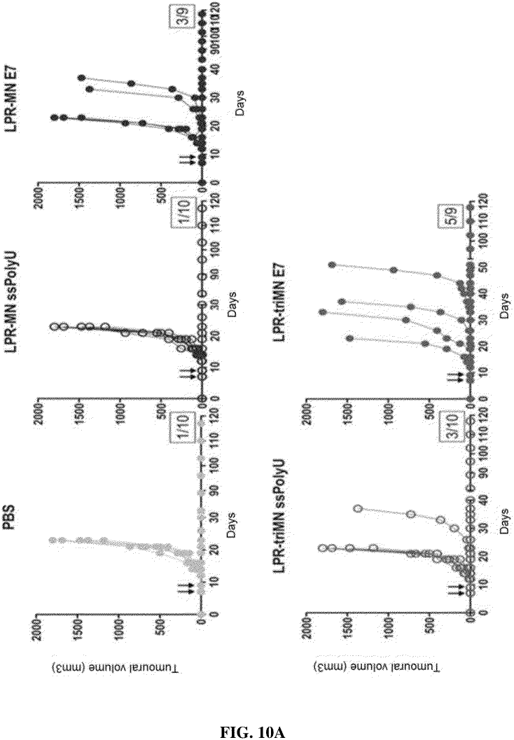

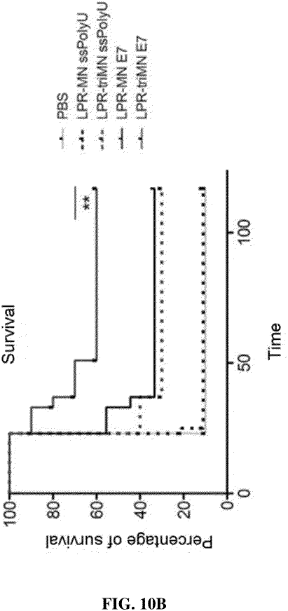

FIG. 10A shows that after 4 months, 5 of the 9 mice vaccinated with LPR-triMN-E7/E7-DC-LAMP were still alive without having developed a tumour. After 4 months, 3 of the 10 mice vaccinated with LPR-triMN-ssPolyU, do not contain encoding mRNA, are also still alive without having developed a tumour. Their survival is apparently attributable to the adjuvant effect of the LPR-triMN that can indeed stimulate the immune response of vaccinated mice. After 4 months, 3 of the 9 mice vaccinated with LPR-MN-E7/E7-DC-LAMP are still alive without having developed a tumour. The injection of PBS or of LPR-MN-ssPolyU does not have any therapeutic effect. FIG. 10B makes it possible to compare the survival of the vaccinated mice with different LPR. The use of LPR-triMN for the vaccination compared to an antigen expressed by the tumour cells has a significant therapeutic effect and prevents the formation of tumours in more than half of the mice vaccinated.

In order to confirm the benefit of LPR-triMN as a therapeutic vaccine, two other tumoural models were tested: the model with melanoma B16F0 (expressing MART1), and the lymphoma model of the cells EG7 expressing OVA. The therapeutic effect of a vaccination with LPR-triMN was evaluated in groups of 10 mice who were administered cells of the tumoural line B16F0 or EG7 by intradermal injection in the ectopic position (left side). The mice were then vaccinated on days 7 and 9 post-injection with PBS, LPR-triMN comprising an RNA ssPolyU or an LPR-triMN comprising an mRNA of MART1 or of OVA respectively.

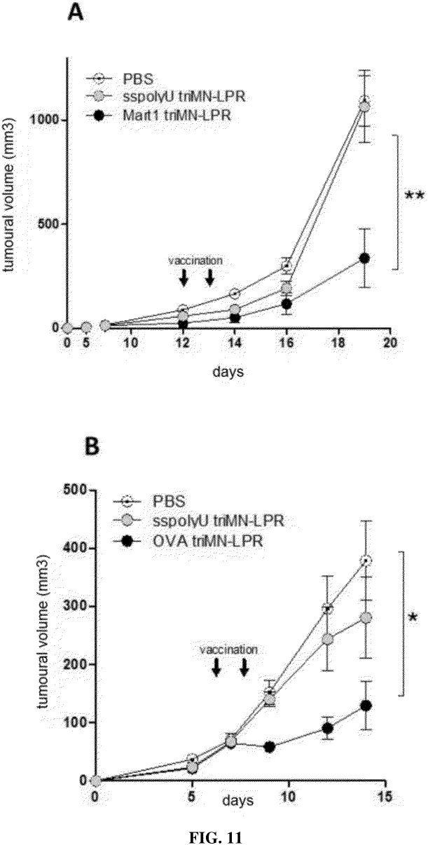

FIG. 11 shows that only the vaccination with LPR-triMN comprising an mRNA of MART1 or of OVA makes it possible to limit the growth of the tumours.

These results show the interest of LPR-triMN for cancer vaccination.

1-15. are (canceled)

16. A compound of formula I: 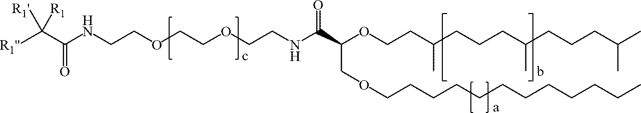
wherein:
a is an integer from 1 to 9;
b is an integer from 1 to 3;
c is an integer from 1 to 130;
R₁, R₁ ′ and R₁″ are identical or different, each one independently representing an H, or a group of the type: 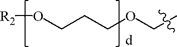 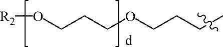 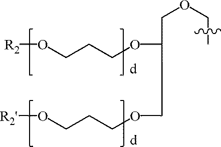
wherein R₂ and R₂ ′ are identical or different, each one independently representing an H or a sugar residue chosen from the list comprising mannose, glucose, fucose, oligomannoses comprising from 2 to 10 mannose units, glycans terminated with a mannose, glycans terminated with a fucose, Lewis-A trisaccharide 3′-sulfate, Lewis-B trisaccharide (Le^{b}), Lewis-X trisaccharide (Le^{x}), tri-N-acetylglucosamine (tri-GlcNAc), PIMs (phosphatidylinositol mannosides), in particular PIM₁ to PIM₆ (phosphatidylinositol mono- to hexa-mannoside), Man9GlcNAc2 oligosaccharide, N-linked oligosaccharides rich in mannose, α-fucose-1-4GlcNAc oligosaccharide, lacto-N-fucopentaose III oligosaccharide containing the Le^{x} trisaccharide, GlcNAc2 Man 3 oligosaccharide, Man 4 oligosaccharides, Manα1-3(Manα1-6)Manα1 oligosaccharide, lipoarabinomannans (LAMs), mannosylated lipoarabinomannans (ManLAMs);
d is an integer from 0 to 5;
with the condition that at least one of the R₂ and R₂ ′ groups is different from H and that R₂ is different
from H when R₂ ′ is absent;
with the condition that at least one of the R₁, R₁ ′ and R₁ ″ groups is different from H.

17. The compound according to claim 16 of formula I: 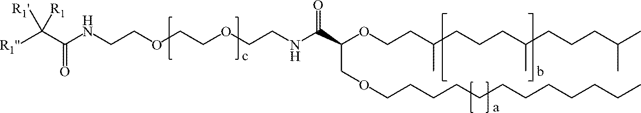
wherein:
a is an integer from 1 to 9;
b is an integer from 1 to 3;
c is an integer from 1 to 130;
R₁, R₁ ′ and R₁″ are identical or different, each one independently representing an H or the group: 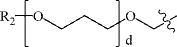 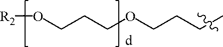 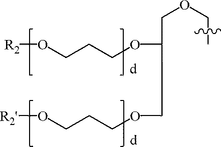
wherein d is an integer from 0 to 5;
wherein R₂ and R₂ ′ are identical or different, each one independently representing mannose, glucose, fucose, or oligomannoses comprising from 2 to 10 mannose units;
with the condition that at least one of the R₁, R₁ ′ and R₁ ″ groups is different from H.

18. The compound according to claim 16 of formula I: 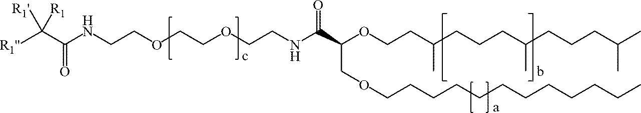
wherein:
a is an integer from 1 to 9;
b is an integer from 1 to 3;
c is an integer from 1 to 130;
R₁, R₁ ′ and R₁′′ are identical or different, each one independently representing an H or the group: 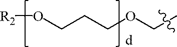 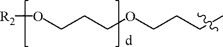 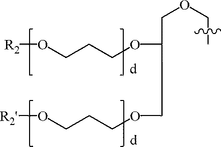
wherein d is an integer from 0 to 5;
wherein R₂ and R₂ ′ represent the group: 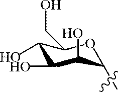
with the condition that at least one of the R₁, R₁ ′ and R₁′′ groups is different from H.

19. The compound according to claim 16 of formula II: 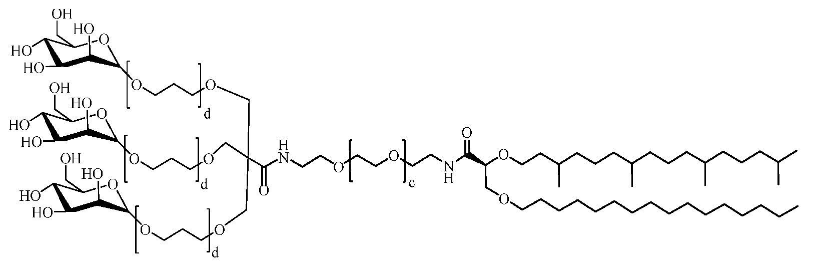 wherein:
c is an integer from 1 to 130;
d is an integer from 1 to 5.

20. The compound according to claim 16 of formula II: 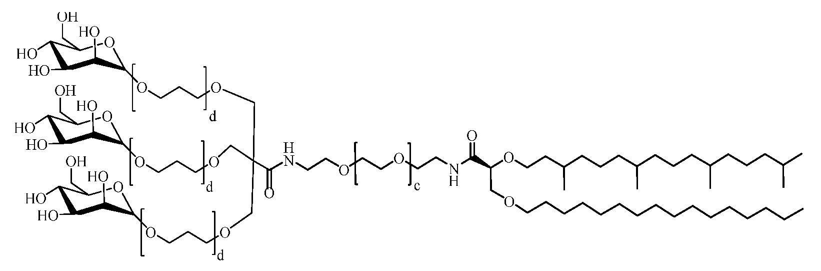 wherein:
c is 5;
d is 2.

21. A liposome comprising a compound of formula I: 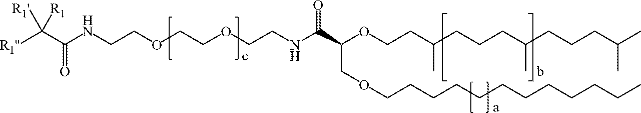
wherein:
a is an integer from 1 to 9;
b is an integer from 1 to 3;
c is an integer from 1 to 130;
R₁, R₁ ′ and R₁′′ are identical or different, each one independently representing an H, or a group of the type: 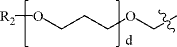 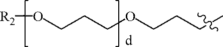 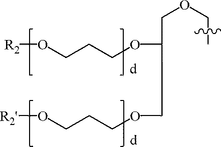
wherein R₂ and R₂′ are identical or different, each one independently representing an H or a sugar residue chosen from the list comprising mannose, glucose, fucose, oligomannoses comprising from 2 to 10 mannose units, glycans terminated with a mannose, glycans terminated with a fucose, Lewis-A trisaccharide 3′-sulfate, Lewis-B trisaccharide (Le^{b}), Lewis-X trisaccharide (Le^{x}), tri-N-acetylglucosamine (tri-GlcNAc), PIMs (phosphatidylinositol mannosides), in particular to PIM₆ (phosphatidylinositol mono- to hexa-mannoside), Man9GlcNAc2 oligosaccharide, N-linked oligosaccharides rich in mannose, α-fucose-1-4GlcNAc oligosaccharide, lacto-N-fucopentaose III oligosaccharide containing the Le^{x} trisaccharide, GlcNAc2 Man 3 oligosaccharide, Man 4 oligosaccharides, Manα1-3(Manα1-6)Manα1 oligosaccharide, lipoarabinomannans (LAMs), mannosylated lipoarabinomannans (ManLAMs);
d is an integer from 0 to 5;
with the condition that at least one of the and R₂ ′ groups is different from H and that R₂ is different from H when R₂′ is absent;
with the condition that at least one of the R₁, R₁′ and R₁″ groups is different from H,
wherein said compound is in proportions ranging from about 1% to about 15% in mole percent with respect to the total number of moles of lipids.

22. The liposome according to claim 21, wherein said compound is of formula II: 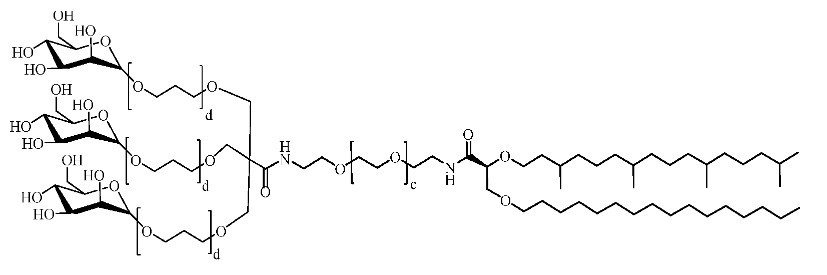 wherein:
c is 5;
d is 2.

23. The liposome according to claim 21, further comprising at least one molecule of interest.

24. The liposome according to claim 21, further comprising at least one molecule of interest, wherein said molecule of interest is able to induce an immune response.

25. The liposome according to claim 21, further comprising at least one molecule of interest, wherein said molecule of interest is a nucleic acid.

26. The liposome according to claim 21, further comprising at least one molecule of interest, wherein said molecule of interest is a cancer-associated antigen selected from the group consisting of: CAP-1, CD 4/m, cell surface proteins of the claudin family CLAUDIN-6, CLAUDIN-18.2 and CLAUDIN-12, c-myc, CT, GnT-V, HAGE, HAST-2, LAGE, NF1, NY-BR-1, proteinase 3, SAGE, SCGB3A2, SCP1, SCP2, SCP3, SSX, SURVrVin, TPI/m, TPTE, CDK4 (cyclin-dependent kinase 4), plS1″1′4′3, p53, AFP, β-catenin, caspase 8, mutated version of p21Ras, Bcr-abl chimera, MUM-I MUM-2, MUM-3, ELF2M, HSP70-2M, HST-2, KIAA0205, RAGE, myosin/m, 707-AP, CDC27/m, ETV6/AML, TEL/Amll, Dekcain, LDLR/FUT, Pml-RARaTEL/AMLI, NY-ESO-I, members of the MAGE family (Melanoma-associated antigen) MAGE-Al, MAGE-A2, MAGE-A3, MAGE-A4, MAGE-A5, MAGE-A6, MAGE-A7, MAGE-A8, MAGE-A9, MAGE-10, MAGE-A11, MAGE-12, MAGE-B, MAGE-C, BAGE, DAM-6, DAM-10, members of the GAGE family (G antigen) GAGE-1, GAGE-2, GAGE-3, GAGE-4, GAGE-5, GAGE-6, GAGE-7B, GAGE-8, NA-88A, CAG-3, RCC-associated antigen G250, oncoproteins E6 and E7 derived from HPV (human papilloma virus), Epstein Barr virus antigens EBNA2-6, LMP-I, LMP-2, gp77, gp100, MART-1/Melan-A, tyrosinase, TRP-I and TRP-2 (tyrosinase-related protein), TRP-2-INT2, PSA, PSM, MClR, ART4, CAMEL, CEA, CypB, HER2/neu, hTERT, hTRT, iCE, Mucl, Muc2, FRAME RU1, RU2, SART-I, SART-2, SART-3, WT and WT1; or is a nucleic acid encoding said cancer-associated antigen.

27. The compound according to claim 16, being a vaccine adjuvant.

28. The liposome according to claim 21, being a vaccine adjuvant.

29. The liposome according to claim 21, being a vaccine.

30. The liposome according to claim 21, being a cancer vaccine.

31. The compound according to claim 16, wherein said compound is comprised within a pharmaceutical composition containing at least one pharmaceutically acceptable excipient.

32. The liposome according to claim 21, wherein said liposome is comprised within a pharmaceutical composition containing at least one pharmaceutically acceptable excipient.

33. A method for vaccinating a subject in need thereof comprising administering to the subject the liposome according to claim 21.

34. The method according to claim 33, wherein said method is for vaccination against cancer.


SEQUENCE LISTING (14 sequences)

SEQ ID NO: 1 - DNA, 20 bp, Artificial Sequence
  CCR7 forward primer
actctccatccaccgaattg

SEQ ID NO: 2 - DNA, 20 bp, Artificial Sequence
  CCR7 reverse primer
cctcatgtcaacctgactgg

SEQ ID NO: 3 - DNA, 23 bp, Artificial Sequence
  CXCR4 forward primer
tccagaatgtgtggtaaattgaa

SEQ ID NO: 4 - DNA, 22 bp, Artificial Sequence
  CXCR4 reverse primer
tcggaatgaagagattatgcag

SEQ ID NO: 5 - DNA, 19 bp, Artificial Sequence
  IL1b forward primer
agttgacggaccccaaaag

SEQ ID NO: 6 - DNA, 20 bp, Artificial Sequence
  IL1b reverse primer
agctggatgctctcatcagg

SEQ ID NO: 7 - DNA, 20 bp, Artificial Sequence
  MMP9 forward primer
ccagaggtaacccacgtcag

SEQ ID NO: 8 - DNA, 22 bp, Artificial Sequence
  MMP9 reverse primer
cttcaagtcgaatctccagaca

SEQ ID NO: 9 - DNA, 23 bp, Artificial Sequence
  PGE2R1 forward primer
tggcttcatattcaagaaaccag

SEQ ID NO: 10 - DNA, 19 bp, Artificial Sequence
  PGE2R1 reverse primer
ggtacacgcgtgactttcg

SEQ ID NO: 11 - DNA, 22 bp, Artificial Sequence
  b-actin forward primer
aagtccctcaccctcccaaaag

SEQ ID NO: 12 - DNA, 21 bp, Artificial Sequence
  b-actin reverse primer
aagcaatgctgtcaccttccc

SEQ ID NO: 13 - DNA, 20 bp, Artificial Sequence
  GAPDH forward primer
gtattgggcgcctggtcacc

SEQ ID NO: 14 - DNA, 22 bp, Artificial Sequence
  GAPDH reverse primer
cgctcctggaagatggtgatgg